SEQUENCE LISTING (36 sequences)

SEQ ID NO: 1 - protein, 16 aa, Artificial
  Synthetic
RTYRTYRTYRTYKKKG

SEQ ID NO: 2 - protein, 12 aa, Artificial
  synthetic
EPIHRSTLTALL

SEQ ID NO: 3 - protein, 5 aa, Artificial
  Synthetic
DDAAG

SEQ ID NO: 4 - protein, 4 aa, Artificial
  Synthetic
DAAG

SEQ ID NO: 5 - protein, 6 aa, Artificial
  Synthetic
HYFKFD

SEQ ID NO: 6 - protein, 6 aa, Artificial
  Synthetic
HFRRHL

SEQ ID NO: 7 - protein, 6 aa, Artificial
  Synthetic
HWRGWV

SEQ ID NO: 8 - protein, 5 aa, artificial
  Synthetic
LLLLL

SEQ ID NO: 9 - protein, 5 aa, Artificial
  Synthetic
DADLE

SEQ ID NO: 10 - protein, 7 aa, Artificial
  Synthetic
AVVWVVE

SEQ ID NO: 11 - protein, 6 aa, Artificial
  Synthetic
VVWVVE

SEQ ID NO: 12 - protein, 7 aa, Artificial
  Synthetic
AVWVEVV

SEQ ID NO: 13 - protein, 8 aa, Artificial
  Synthetic
ADRASPYE

SEQ ID NO: 14 - protein, 7 aa, Artificial
  Synthetic
AVVWVVK

SEQ ID NO: 15 - protein, 7 aa, Artificial
  Synthetic
AVVWVVK

SEQ ID NO: 16 - protein, 5 aa, Artificial
  Synthetic
VVWVV

SEQ ID NO: 17 - protein, 6 aa, Artificial
  Synthetic
DRASPY

SEQ ID NO: 18 - protein, 5 aa, Artificial
  Synthetic
WFRHY

SEQ ID NO: 19 - protein, 6 aa, Artificial
  Synthetic
VVWVVE

SEQ ID NO: 20 - protein, 7 aa, Artificial
  Synthetic
AVVWVVE

SEQ ID NO: 21 - protein, 7 aa, Artificial
  Synthetic
DRASPYE

SEQ ID NO: 22 - protein, 8 aa, Artificial
  Synthetic
ADRASPYE

SEQ ID NO: 23 - protein, 7 aa, Artificial
  Synthetic
AVWVEVV

SEQ ID NO: 24 - protein, 7 aa, Artificial
  Synthetic
AWFRHYE

SEQ ID NO: 25 - protein, 6 aa, Artificial
  Synthetic
AVVWVV

SEQ ID NO: 26 - protein, 7 aa, Artificial
  Synthetic
ADRASPY

SEQ ID NO: 27 - protein, 8 aa, Artificial
  Synthetic
AVWVEVVV

SEQ ID NO: 28 - protein, 6 aa, Artificial
  Synthetic
AWFRHY

SEQ ID NO: 29 - protein, 9 aa, Artificial
  Synthetic
AADRASPYE

SEQ ID NO: 30 - protein, 7 aa, Artificial
  Synthetic
AVVWVVK

SEQ ID NO: 31 - protein, 8 aa, Artificial
  Synthetic
ADRASPYK

SEQ ID NO: 32 - protein, 8 aa, Artificial
  Synthetic
AMWFPHYK

SEQ ID NO: 33 - protein, 7 aa, Artificial
  Synthetic
AMWFPHY

SEQ ID NO: 34 - protein, 8 aa, Artificial
  Synthetic
AMWFPHYK

SEQ ID NO: 35 - protein, 6 aa, Artificial
  Synthetic
VVWVVE

SEQ ID NO: 36 - protein, 5 aa, Artificial
  Synthetic
VWVVE

(19) United States
(12) Patent Application Publication
(10) Pub. No.: US 2013/0310265 A1
(43) Pub. Date: Nov. 21, 2013

(54) METHODS OF PREPARING CYCLIC PEPTIDES AND USES THEREOF

(71) Applicant: NORTH CAROLINA STATE UNIVERSITY, RALEIGH, NC (US)

(72) Inventors: STEFANO MENEGATTI, WILLOW SPRING, NC (US); RUBEN CARBONELL, RALEIGH, NC (US); AMITH D. NAIK, RALEIGH, NC, NC (US); KEVIN BLACKBURN, CLINTON, NC (US)

(73) Assignee: NORTH CAROLINA STATE UNIVERSITY, RALEIGH, NC (US)

(21) Appl. No.: 13/826,949

(22) Filed: Mar. 14, 2013

Related U.S. Application Data
(60) Provisional application No. 61/613,722, filed on Mar. 21, 2012.

(51) Int. Cl. C07K 1/04

(52) U.S. Cl. CPC C07K 1/047
USPC 506/6; 506/30; 506/9; 506/2

(57) ABSTRACT

This invention is directed to the discovery of improved methods of preparing cyclic peptides, cyclic peptide esters, cyclic peptide amidines, and libraries of these compounds. The invention also includes uses of these compounds and libraries for screens as drugs and binders of biologics.

### CROSS REFERENCE TO RELATED APPLICATIONS

This application claims the benefit of 61/613,722 filed Mar. 21, 2012, Menegatti et al., entitled “Methods of Preparing Cyclic Peptides and Uses Thereof” having Atty. Docket No. NS12001USV, which is hereby incorporated by reference in its entirety.

### 1. FIELD OF THE INVENTION

This invention relates generally to the discovery of improved methods for the synthesis of cyclic peptides, cyclic depsipeptides, cyclic peptide amidines, and libraries thereof. The invention also includes uses of these compounds and libraries for the identification of biomimetics, affinity ligands, and drugs.

### 2. BACKGROUND OF THE INVENTION

2.1. Small Ligands and Peptides in Medicine and Affinity Chromatography

Small synthetic compounds with high specificity and affinity for biomolecules have great potential for a broad range of applications, from cell biology, to medicine, to the purification of protein therapeutics. Small synthetic peptides in particular are an extremely promising class of compounds. Owing to their selectivity, low toxicity and immunogenicity, the possibility of structural modifications and incorporating non-natural moieties to enhance protease resistance and bioavailability, these compounds have found increasing application in medicine, as both diagnostics and therapeutics. Many therapeutic peptides are today available for the treatment of a vast array of diseases, such as diabetes, obesity, Crohn's disease, osteoporosis, cancer, cardiovascular disease, acromegaly, enuresis, etc.

Furthermore, over the past 20 years, small peptides have been proposed as affinity ligands for the purification of biomolecules. A number of advantageous characteristics, such as chemical stability, mild elution conditions, no immunogenicity, and low production costs have drawn increasing attention to peptide ligands and indicate that these compounds could replace the currently used biological ligands in both analytical and preparative aspects of downstream processes in biomanufacturing. Considerable effort has been paid particularly in the area of antibody purification to identify peptide ligands in replacement of Protein A and Protein G. As these protein ligands, although highly specific, suffer from immunogenicity, harsh elution conditions, and high cost, small peptides have been indicated as potential alternatives. Several linear peptides have in fact been reported as affinity ligands for antibody purification, such as the sequences (RTY)₄K₂KG (TG19318), EPIHRSTLTALL, the peptide based D₂AAG and DAAG (SEQ ID NO:1-4), and three highly homologous hexapeptide ligands HYFKFD, HFRRHL, and HWRGWV (SEQ ID NO:5-7).

Finally, small synthetic peptides have found application as alternatives to antibodies in sensing and diagnostic applications. Peptide-based ELISA tests have been designed for the detection of viruses and other pathogens and increasing research is ongoing to extend the use of these compounds as capture agents and detecting tools.

2.2. Cyclic Peptides as Biomimetics, Drugs and Affinity Ligands

In the vast scenario of small synthetic peptides, sterically constrained (cyclic) peptides hold a particularly relevant position. Due to their conformational rigidity these compounds show superior properties compared to linear peptides, in particular: 1) higher specificity and avidity towards the target, 2) higher enzymatic stability, and 3) higher conformational integrity. Natural cyclic peptides play a role in the control of protein expression, enzyme inhibition and viral activity, and possess specific antibiotic functions and membrane penetration powers. Many natural cyclic peptides, such as hormones (somatostatin and oxytocin), antibiotics (gramicidin), immunosuppressants (cyclosporine), cancer chemotherapeutics (actinomycin D), antifungal agent (capsofungin), and toxins (amanitins) are already extensively employed therapeutic agents. Furthermore, research is ongoing towards the discovery of highly potent cyclic peptide based drugs. Ocreotide is a well-known example of synthetic hormone that mimics somatostatin as antagonist of growth hormone and insulin, yet being more potent than its natural counterpart.

In addition to drug discovery, cyclic peptides have been proposed as affinity ligands for the purification of biomolecules. Krook et al. developed a cyclic nonapeptide with high affinity for chymotrypsin. Krook M., Lindbladh C., Eriksen J. A. and Mosbach K. (1998) Selection of a cyclic nonapeptide inhibitor to alpha-chymotrypsin using a phage display peptide library, Mol. Divers. 3, 149-159. Millward et al. designed a cyclic binding peptide (cycGiBP) that targets a signaling protein Gail with antibody-like affinity and high proteolytic stability. Millward S. W., Fiacco S., Austin R. J. and Roberts R. W. (2007) Design of cyclic peptides that bind protein surfaces with antibody-like affinity, Chem. Biol. 2, 625-634. Gaj et al. have developed a cyclic peptide affinity ligand for avidin and neutravidin. Gaj T., Meyer S. C., Ghosh I. (2007) The AviD-tag, a NeutrAvidin/avidin specific peptide affinity tag for the immobilization and purification of recombinant proteins, Protein Expr. Purif. 56, 54-61. This ligand binds the avidin and neutravidin with high affinity, and is used as an affinity tag for purification of recombinant proteins. Zhang et al. identified two cyclic peptides for Tensin. Zhang Y., Zhou S., Wavreille A., DeWille J. and Pei D. (2008) Cyclic Peptidyl Inhibitors of Grb2 and Tensin SH2 Domains Identified from Combinatorial Libraries, J. Comb. Chem., 10, 247-255.

2.3. Methods of Cyclization

Cyclic peptide ligands can be obtained in a variety of ways, some of them employing only natural amino acids and some others based on the use of non-natural amino acids. Akaji K., Kiso Y. (2003) Synthesis of cystine peptides, In Houben-Weyl, Methods of organic chemistry, volE22b, Synthesis of peptides and peptidomimetics, Goodman M., Felix A., Moroder L., Toniolo C. (Eds), Georg Thieme Verlag, Stuttgart, 101-141. Among the former, the best-known approaches are the formation of disulfide bridges between two cysteine residues and the end-to-tail, side chain-to-tail, or side chain-to-side chain cyclization reactions between the peptide C-terminus and N-terminus, the glutamic or aspartic acid and lysine residues. Romanovskis P. and Spatola A. F. (1998) Preparation of head-to-tail cyclic peptides via side-chain attachment: implications for library synthesis, J. Peptide Res. 52, 356-374; Blackburn C. and Kates S. A. (1997) Solid-phase synthesis of cyclic homodetic peptides, Methods Enzymol., vol. 289, 175-198. The disulfide bond, however, is labile in reducing environment and this poses a severe limitation to such compounds in their application as drugs or affinity ligands.

Cyclization methods using non-natural amino acids include the Ring Closing Metathesis (RCM) method and the Click Chemistry approach. For RCM see: Grubbs R. H., Miller S. J. and Fu G. C. (1995) Ring-closing metathesis and related processes in organic synthesis, Acc. Chem. Res. 28, 446-452; Scholl M., Trnka T. M., Morgan J. P. and Grubbs R. H. (1999) Increased ring closing metathesis activity of ruthenium-based olefin metathesis catalysts coordinated with imidazolin-2-ylidene ligands, Tetrahed. Lett. 40, 2247-2250. For Click Chemistry see: Kolb H. C., Finn M. G. and Sharpless K. B. (2001), Click chemistry: diverse chemical function from a few good reactions, Angew. Chem. Ing. Ed. 40, 2004-2021; Kolb H. C. and Sharpless K. B. (2003), The growing impact of click chemistry on drug discovery, Drug Discovery Today 8, 1128-1137; Moses J. E. and Moorhouse A. D. (2007), The growing applications of click chemistry, Chem. Soc. Rev. 36, 1249-1262. RCM refers to the intramolecular olefin metathesis catalyzed by a Ruthenium-based Grubbs' reagent between two allyl glycines located at the ends of the peptide. Milles S. J., Blackwell H. E. and Grubbs R. H. (1996), Application of ring-closing metathesis to the synthesis of rigid amino acids and peptides, J. Am. Chem. Soc. 118, 9606-9614; Reichwein J. F., Versluis C. and Liskamp R. M. J. (2000), Synthesis of cyclic peptides by ring-closing metathesis, J. Org. Chem. 65, 6187-6195; Kazmaier U., Hebach C., Watzke A., Maier S., Mues H. and Huch V. (2005), A straightforward approach towards cyclic peptides via ring-closing metathesis—scope and limitations, Org. Biomol. Chem. 3, 136-145.

The click chemistry consists of a Huisgen cycloaddition between the alkyne and the azide residues of non-natural amino acids, such as propargylglycine and any azido amino acid, leading to a triazole link. Turner R. A., Oliver A. G. and Lokey R. S. (2007), Click chemistry as a macrocyclization tool in the solid-phase synthesis of small cyclic peptides, Org. 9, 24, 5011-5014; Jagasia R., Holub J. M., Bollinger M., Kirshenbaum K. and Finn M. G. (2009), Peptide cyclization and cyclodimerization by CuI-mediated azide-alkyne cycloaddition, J. Org. Chem. 74, 2964-2974. RCM, however, suffers from drawbacks such as long reaction times and moderate yields of cyclic peptides, along with high catalyst loading and difficult removal of ruthenium impurities upon completion of reaction. Robinson A. J., Elaridi J., Van Lierop B. J., Mujcinovic S. And Jackson W. R. (2007), Microwave-assisted RCM for the synthesis of carbocyclic peptides, J. Pept. Sci. 13, 280-285. The major limitation of click chemistry is the high cost of the non-natural amino acids.

2.4. Solid Phase Combinatorial Libraries of Cyclic Peptides

The screening of solid-phase combinatorial libraries of cyclic peptides is a powerful technique for the identification of novel cyclic peptide ligands and biomimetics. Lam, K. S., Lebl, M., and Krchnak, V. (1997) The “one-bead-one-compound” combinatorial library method. Chem. Rev. 97, 411-448; Wang, G., De, J., Schoeniger, J. S., Roe, D. C. and Carbonell, R. G. (2004) A hexamer peptide ligand that binds selectively to staphylococcal enterotoxin B: isolation from a solid phase combinatorial library. J. Pept. Res. 64, 51-64; Lam, K. S., Salmon, S. E., Hersh, E. M., Hruby, V. J., Kazmierski, W. M. & Knapp, R. J. (1991). A new type of synthetic peptide library for identifying ligand-binding activity. Nature, 354, 82-84. However, the process of ligand discovery is hindered at the stage of sequence identification. In fact, while the sequencing of a linear peptide is routinely carried out with high level of precision by Edman degradation or single stage of MS/MS, the determination of a cyclic sequence is much more challenging. Joo S. H., Xiao Q., Ling Y., Gopishetty B. and Pei D. (2006), High-throughput sequence determination of cyclic peptide library members by partial edman degradation/mass spectrometry, J. Am. Chem. Soc. 128, 13000-13009. Edman degradation is not possible with cyclic peptides due to the absence of the peptide N_{α}-terminus. Although MS-based techniques have been reported for the sequencing of cyclic peptides, they entail considerable effort and high level of uncertainty. In a mass spectrometer, in fact, the cyclic peptide undergoes ring opening at multiple positions to produce a complex mixture of shorter peptides, making spectral interpretation difficult and highly uncertain. Joo (2006); Eckert K., Schwarz H., Tomer K. B., and Gross M. L. (1985), Tandem mass spectrometry methodology for the sequence determination of cyclic peptides, J. Am. Chem. Soc. 107, 6765-6769; Ngoka L. C. and Gross M. L. (1999) Multistep tandem mass spectrometry for sequencing cyclic peptides in an ion-trap mass spectrometer, J. Am. Soc. Mass. Spectrom. 10, 732-746; Schilling B., Wang W., McMurray J. S, and Medzihradszky K. F. (1999), Fragmentation and sequencing of cyclic peptides by matrix-assisted laser desorption/ionization postsource decay mass spectrometry, Rapid Commun, Mass Spectrom. 13, 2174-2179; Lin S., Liehr S., Cooperman B. S, and Cotter R. J. (2001), Sequencing cyclic peptide inhibitors of mammalian ribonucleotide reductase by electrospray ionization mass spectrometry, J. Mass. Spectrom. 3, 6, 658-663; Redman J. E., Wilcoxen K. M. and Ghadiri M. R. (2003), Automated mass spectrometric sequence determination of cyclic peptide library members, J. Comb. Chem. 5, 33-40. Extensive research has been performed and is ongoing on different MS techniques, such as electrospray ionization (ESI-MS) with collision-induced dissociation (CID), MALDI post-source decay (MALDI-PSD) and MALDI-TOF/TOF, to provide dependable sequencing methods.

Some strategies have been proposed to circumvent the difficulties involved in the post screening hit identification. Bak et al. reported esterase-sensitive cyclic prodrugs containing an (acyloxy)alkoxy linker (Bak et al. 1999, J Peptide Res 53 393-202). Specifically, they disclose cyclic prodrugs of opioid peptides [Leu5]-enkephalin and DADLE (Tyr-D-Ala-Gly-Phe-D-Leu-OH) (SEQ ID NO: 8-9). Their data suggests these prodrugs have higher permeability across a cell membrane than the linear opioid peptides. This method, however, is extremely laborious and is not applicable to a procedure of sequence identification of the linearized peptides selected from a library screening.

Liu et al. recently reported cyclic peptides that bind human prolactin receptor identified from a one-bead-two-peptide (OBTP) methodology (Liu et al. 2009, Bioorg Med Chem 17 1026-1033). This method entails the segregation of each library bead into an outer layer, which is accessible to the target protein and contains the peptides in cyclical form, and an inner core, containing the linear precursor of the peptide. After selecting the beads that bind the target protein through the cyclic peptides in the outer layer, the peptide sequences are determined by partial Edman degradation/mass spectrometry (PED/MS) of the internal linear precursors. Although successful, this method requires a long treatment of modification of the resin prior to peptide synthesis.

Lee et al. recently reported a methodology for the construction of libraries of non-natural peptoid cyclized by formation of alkylthioaryl bridge (Lee et al. 2010 Chem Comm 46 8615-8617). Specifically, the peptoid is cyclized by reacting the thiol group of cysteine and a cyanuric chloride moiety located on the opposite end of the peptoid chain. The library of these thioaryl-bridged cyclic peptoids can be used for drug screening. For identification of binders, the cyclic peptoid is linearized by oxidizing the thioether bond to a sulfone, using m-chloroperoxybenzoic acid, followed by hydrolysis with 1M sodium hydroxide for 12 h. The linearized peptoids are finally sequenced by MS/MS. This method would not be applicable to libraries of natural amino acids, as the reaction with a strong oxidizing agent like m-chloroperoxybenzoic acid would irreversibly oxidize the functional groups of many amino acids residues, e.g., serine, lysine, histidine, tryptophan. The presence of these oxidized amino acids would vastly complicate the interpretation of subsequent sequencing results by MS or other methods.

The method proposed in this disclosure does not involve either the formation of weak disulfide bonds, or the use or costly non-natural amino acids, or any guesswork in spectral analysis, or any consuming iterative deconvolution.

### 3. SUMMARY OF THE INVENTION

In one embodiment, the invention is directed to a method for synthesizing a cyclic peptide ligand with selectivity and affinity for a biologic of interest which comprises:
(a) synthesizing a solid-phase library of reversible cyclic heterodetic peptides;
(b) selecting a reversible cyclic heterodetic peptide that shows selectivity and affinity for the biologic of interest;
(c) linearizing and sequencing the selected reversible cyclic heterodetic peptide; and
(d) solid-phase synthesizing a cyclic peptide ligand with a sequence corresponding to the selected reversible cyclic heterodetic peptide.

In another embodiment, the invention is directed to a method for synthesizing a cyclic depsipeptide which comprises:
(a) coupling a protected tri-functional molecule with a plurality of protecting groups onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) cleaving a protecting group from either (i) the protected amino acid or peptide, or (ii) the tri-functional molecule so as to form a deprotected amino acid or peptide, or a deprotected tri-functional molecule coupled on the solid support;
(e) coupling a protected cleavable linker with either (iii) the deprotected amino acid or peptide, or (iv) the deprotected tri-functional molecule;
(f) cleaving the protecting group from the cleavable linker and a protecting group from either (iii) the protected amino acid or peptide, or (iv) the protected tri-functional molecule and cyclizing so as to form a cyclic depsipeptide coupled on the solid support; and
(g) cleaving any remaining protecting groups from the cyclic depsipeptide coupled on the solid support.

In yet another embodiment, the invention is directed to a method for synthesizing a cyclic amidine-peptide, the method comprising:
(a) coupling a protected tri-functional molecule onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) deprotecting a primary amino group from the protected amino acid or peptide and a primary amino group from the tri-functional molecule so as to form a deprotected amino group on the acid or peptide and a deprotected amino group on the tri-functional molecule coupled on the solid support;
(e) reacting a bis-imidoester linker with the primary amino group on the acid or peptide and the primary amino group on the tri-functional molecule coupled on the solid support so as to form an cyclic amidine-peptide coupled on the solid support; and
(f) cleaving any remaining protecting groups from the cyclic amidine-peptide coupled on the solid support.

The invention is also directed to a solid-phase library of cyclic depsipeptides or cyclic amidine-linked peptides wherein each depsipeptide or cyclic amidine-linked peptide independently has the structure:

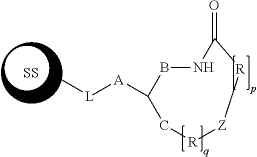

a. A, B, and C, are independently C₁₋₈ alkyl, C₂₋₈ alkenyl, C₃₋₈ alkynyl, or C₁₋₈ alkoxy;
b. p and q are independently integers 0-30 with the proviso that the sum of p and q is greater than 2;
c. each R is independently a biomonomer;
d. L is a suitable linker to the solid support;
e. Z is an ester bond [—CO—O—] or [—O—CO—]; or —(CH₂)_{y}(NH(Ac))(CH₂)ₓCO—O—, —(CH₂)_{y}(NHCO)(CH₂)ₓCO—O—, —(CH₂)_{y}(H(Ac))(CH₂)ₓ)OC—O—, —(CH₂)_{y}(NHCO)(CH₂)ₓOC—O—, —CO—O—(CH₂)ₓ(NH(Ac))(CH₂)_{y}—, —CO—O(CH₂)ₓ(NHCO)(CH₂)_{y}—, —OCO(CH₂)ₓ(NH(Ac))(CH₂)_{y}—, —OCO(CH₂)ₓ(NHCO)(CH₂)_{y}—; or an amidine bond —C(═NH)—NH— or —NHC(═NH);
   —(CH₂)_{y}(NH(Ac))(CH₂)ₓC(═NH)—NH—, —(CH₂)_{y}(NHCO)(CH₂)ₓC(═NH)—NH, —(CH₂)_{y}(NH(Ac))(CH₂)ₓNHC(═NH)—, —(CH₂)_{y}(NHCO)(CH₂)ₓNHC(═NH), —C(═NH)—NH(CH₂)ₓ(NH(Ac))(CH₂)_{y}—, —C(═NH)—NH(CH₂)ₓ(NHCO)(CH₂)_{y}—, —NHC(═NH)(CH₂)ₓ(NH(Ac))(CH₂)_{y}—, —NHC(═NH)(CH₂)ₓ(NHCO)(CH₂)_{y}—; and
f. x is an integer from 1-8 and y is an integer from 0-8.

### 4. BRIEF DESCRIPTION OF THE FIGURES

FIG. 1 shows the formation of amidines, their hydrolysis and ammonolysis reactions.

FIG. 2 shows examples of amino dicarboxylic tri-functional molecules.

FIG. 3 shows examples of diamino acids tri-functional molecules.

FIG. 4 shows examples of carboxyl-protected α-, β-, or γ-hydroxy acids suitable for cleavable linkers.

FIG. 5 shows examples of ester forming cleavable linkers, N,O-protected hydroxy amino acids.

FIG. 6 shows examples of acetoxyacetic acid or acetoxy propionic acid as cleavable linkers.

FIG. 7 shows examples of ester forming cleavable linkers using mono-protected dicarboxylic acids.

FIG. 8 shows examples of ester containing heterobifunctional cleavable linkers.

FIG. 9 shows additional examples ester containing heterobifunctional cleavable linkers where the ester of an amino acid and a C-protected hydroxy acid

FIG. 10 shows a method for the synthesis of cyclic depsipeptides.

FIG. 11 shows allyl lactate and its structural analogue alanine allyl ester.

FIG. 12 shows a cyclic depsipeptide and the corresponding homodetic cyclic peptide.

FIG. 13 shows an example of a dicarboxylic acid as tri-functional molecule and a hydroxyl-protected ester forming cleavable linker for the synthesis of cyclic depsipeptides.

FIG. 14 shows an example of a depsipeptide cyclized by formation of amide bond between the free carboxyl group of glutamic acid and the amino group of the additional amino acid.

FIG. 15 shows the corresponding homodetic cyclic peptide when the cleavable linker is replaced by a non-cleavable linker.

FIG. 16 shows examples of diaminobutanoic acid non-cleavable linkers.

FIG. 17 shows an example of a homodetic cyclic peptide.

FIG. 18 shows an example with a diamino acid as tri-functional molecule and a carboxyl-protected ester forming cleavable linker are used for the synthesis of cyclic depsipeptides.

FIG. 19 shows an example of the homodetic cyclic peptide corresponding to the depsipeptide of FIG. 18.

FIG. 20 shows an example with a dicarboxylic acid as tri-functional molecule and the ester of a N-protected amino acid and a hydroxy acid as ester containing cleavable linker are used for synthesis of cyclic depsipeptides.

FIG. 21 shows an example with a dicarboxylic acid as tri-functional molecule and the ester of a carboxyl-protected hydroxy acid and an amino acid as ester containing cleavable linker used for the synthesis of cyclic depsipeptides.

FIG. 22 shows the homodetic cyclic peptide analogue corresponding to the FIG. 21 depsipeptide.

FIG. 23 shows an example where a diamino acid as tri-functional molecule and the ester of a dicarboxylic acid and a carboxyl-protected hydroxy acid as ester containing cleavable linker are adopted for the synthesis of cyclic depsipeptides.

FIG. 24 shows the homodetic cyclic peptide analogue corresponding to the FIG. 23 depsipeptide.

FIG. 25 shows an example where a diamino acid as tri-functional molecule and a homobifunctional imidoester crosslinker are adopted for the synthesis of reversible cyclic amidine-peptides.

FIG. 26 shows the homodetic cyclic peptide analogue corresponding to the FIG. 25 amidine-linked cyclic peptide.

FIG. 27 illustrates different locations of the “key stone” tri-functional amino acid (glutamic acid in the figure) along a linear peptide chain and the corresponding cyclic depsipeptides.

FIG. 28 shows the RP-HPLC (C18) to plots demonstrating the purity of the depsipeptides on solid-phase.

FIG. 29 shows the structure and the ESI-MS/MS analysis of linearized sequences Lac-A-VVWVV-E and Lac-VVWVV-E (SEQ ID NO:10-11) cleaved from a single bead.

FIG. 30 shows the structure and the ESI-MS/MS analysis of linearized sequence Lac-A-VWV-E-VV (SEQ ID NO:12) from a single bead

FIG. 31 shows the structure of Lac-A-VWV-E-VV (SEQ ID NO:12).

FIG. 32 shows the structure and the ESI-MS/MS analysis of linearized sequence Lac-A-DRASPY-E (SEQ ID NO:13) from a single bead.

FIG. 33 shows RP-HPLC results for the cyclic amidine-peptide sequence cyclo[AVVWVVK-Adipimidate] (SEQ ID NO:14).

FIG. 34 shows the structure and the ESI-MS/MS analysis of linearized sequences AVVWVVK (SEQ ID NO:15) cleaved from a single bead.

FIG. 35 shows the matching binding chromatograms for the cyclic depsipeptide and the cyclic peptide version of the same ligand sequence.

FIG. 36a-36g shows a general synthetic strategy for the synthesis of reversible cyclic depsipeptide. CM stands for ChemMatrix®.

### 5. DETAILED DESCRIPTION OF THE INVENTION

This invention is directed to the synthesis of combinatorial libraries of a new family of constrained (cyclic) peptides synthesized by a novel chemistry that simplifies the sequence identification of the leads selected by library screening. These novel compounds may find a wide number of applications, including drug discovery, proteomics, in-line process sensors, on-the-field medical diagnostics, pathogen detection and removal for homeland security, and identification of affinity ligands for the purification of biologicals from complex mixtures.

The disclosure presents methods of solid-phase synthesis of reversible cyclic peptides and libraries thereof. This method comprises the incorporation of a cleavable linker in a known position within the peptide sequence prior to cyclization. The linker allows one to open the cyclic molecule and return the peptide to its linear structure. As the proposed cleavable linkers are structurally and chemically analogue to natural amino acids or mixtures thereof, after ring opening the resulting linearized molecule is highly similar or identical to a linear peptide and can be sequenced with techniques routinely employed for the sequencing of such compounds, such as single stage MS/MS or Edman degradation. When needed, the treatment used for ring opening can also allow the release of the linearized peptide from the solid support to the liquid phase, thereby making it available for other analysis that can further substantiate the process of sequence identification. The method applies to the synthesis of combinatorial libraries that can be screened against biologicals for the easy and high throughput identification of biomimetics, affinity ligands or drugs. These libraries have not been reported in literature, at least for the purpose of ligand or drug identification. To the best of our knowledge the synthesis of cyclic peptides that can be returned to their linear structure owing to the presence of a cleavable linker within the sequence has not been yet reported.

More specifically, the key features of the proposed reversible cyclic peptides are (1) a “key stone” tri-functional molecule and (2) a cleavable linker. The former is a Y-shaped amino acid upon which the peptide cycle is articulated. The latter is a bifunctional molecule, analogue in structure and chemical properties to a single protected amino acid or to a protected compound of two or more amino acids. The most notable and important characteristic of the reversible cyclic heterodetic peptides proposed in this invention is that they are similar, under the aspects of structural and chemical properties and binding behavior, to their corresponding cyclic homodetic peptide analogues. The fulfillment of this key requirement guarantees that the identified sequences, when employed in their cyclic homodetic peptide form, present the same behavior of target binding/interaction as their reversible cyclic heterodetic precursors by means of which they have been identified. Furthermore, due to their structural and chemical similarity to cyclic homodetic peptides, the ring opening of the reversible cyclic heterodetic peptides result in linearized molecules highly similar or identical to linear homodetic peptides and can therefore be easily sequenced with techniques routinely employed for the sequencing of such compounds.

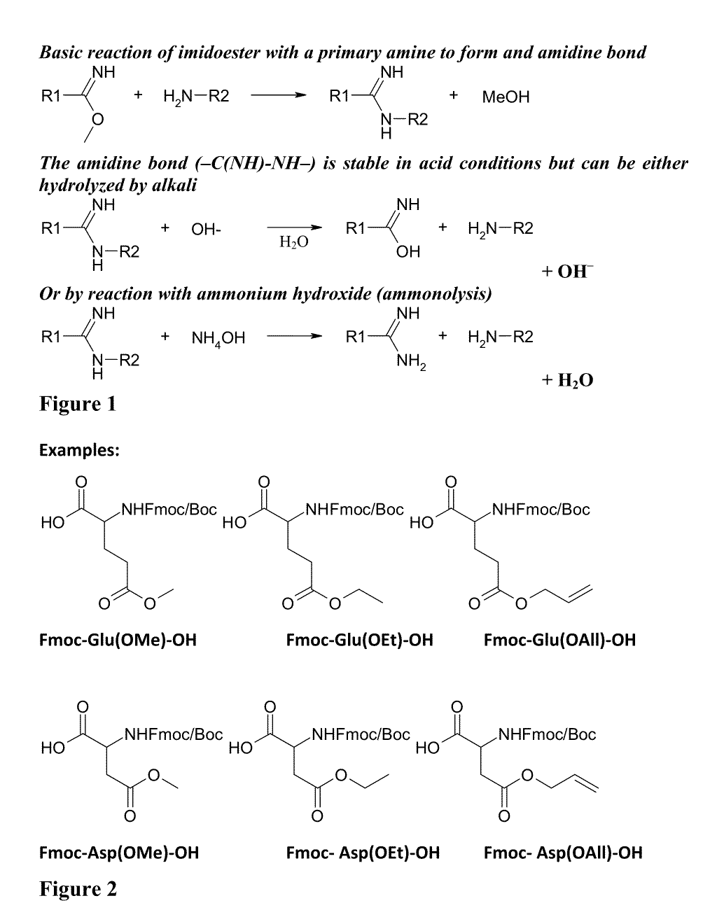

One of ordinary skill in the art would recognize that a broad range of cleavable bond exist in organic chemistry and, more particularly, in peptide chemistry. Without limiting the scope of this invention, this disclosure proposes the use of two classes of cleavable bonds, namely ester and amidine, which are both hydrolyzed in alkaline conditions. Ester bonds are formed by reaction between an oxoacid and a hydroxyl compound, and more commonly by condensing a carboxylic acid with an alcohol. Ester bonds are readily hydrolyzed in alkaline conditions to return the oxoacid and the hydroxyl compounds. Linear or cyclic peptides in which one or more peptide bond (—CO—NH—) is replaced by ester bond(s) (—CO—O—) are respectively referred as linear or cyclic depsipeptides. Amidine bonds are formed by reaction between an imidoester group and a primary amino group. As literature indicates, an imidoester group reacts specifically with α-amino groups in a pH range of 8.0-8.5 and also with ε-amino groups at higher pH values, 9.5-10.00 (FIG. 1). While stable in acid conditions, amidine bonds are stable in acid conditions but are cleaved by acqueous strong alkali or ammonia, to restore the original amino groups.

Linear or cyclic heterodetic peptides in which one or more peptide bond is replaced by an amidine bond will be referred in this invention as linear or cyclic amidine-peptides.

Without wishing to limit the scope of the invention, this disclosure presents the use of two classes of linkers: (1) ester containing and ester forming linkers for making cyclic depsipeptides and (2) imidoester crosslinkers for cyclic amidine-peptides. A subset of the first group are linkers that form an ester linkage using an amino acid side chain, such the —OH group in serine. Other suitable side chain-to-tail ester forming linking amino acids are tyrosine, hydroxy proline, hydroxyvaline, or hydroxyleucine.

For each method of synthesis of reversible heterodetic cyclic peptides, this invention presents methods for the synthesis of the corresponding irreversible homodetic cyclic peptide forms. In analogy to the above listed cleavable linkers, these methods employ two classes of uncleavable linkers, respectively (1) amide-containing and amide-forming linkers and (2) succinimide ester crosslinkers for cyclic amidine-peptides.

Furthermore, the proposed technique is also open to a wide range of chemical and spatial diversity. In fact, it allows the incorporation of non-natural peptides and peptoids in the sequence, provided that the selection of these building blocks allows the unequivocal sequencing by MS/MS and, when possible, by Edman degradation. The conformational diversity can be obtained by judiciously varying the position of the “key stone” amino acid and the length of the peptide sequence to produce different combinations of the linear vs. cyclic portions within the same molecule. This broadens the conformational ability of these compounds to bind targets and enhances the “affinity” nature/behavior of these molecules.

Without limiting the scope of the invention, this disclosure proposes the adoption of a radiological approach for screening, as presented by Mondorf and Carbonell, and of a bioinformatics approach for sequence determination from spectral data obtained by MS/MS analysis of the linearized sequences. K. Mondorf, D. B. Kaufman, and R. G. Carbonell, Screening of Combinatorial Peptide Libraries: Identification of Ligands for Affinity Purification of Proteins Using a Radiological Approach, Journal of Peptide Research, 6, 526-536. The bioinformatic approach is proposed in order to perform an unbiased determination of the peptide sequences from the selected beads. The method employs the software Mascot to compare the spectral data obtained by MS/MS analysis with a library of theoretical spectra generated according to model fragmentation patterns. The selection of the sequences from the list of possible matches provided by the software is to be based on considerations of peptide composition and consensus homology.

In particular non-limiting embodiments, the present invention provides In one embodiment, the invention is directed to a method for synthesizing a cyclic peptide ligand with selectivity and affinity for a biologic of interest which comprises:
(a) synthesizing a solid-phase library of reversible cyclic heterodetic peptides;
(b) selecting a reversible cyclic heterodetic peptide that shows selectivity and affinity for the biologic of interest;
(c) linearizing and sequencing the selected reversible cyclic heterodetic peptide; and
(d) solid-phase synthesizing a cyclic peptide ligand with a sequence corresponding to the selected reversible cyclic heterodetic peptide.

In the embodiment above, the reversible cyclic heterodetic peptide may be a cyclic depsipeptide or a cyclic amidine-peptide. In a preferred embodiment, a plurality of cyclic peptide ligands are synthesized.

In another embodiment, the invention is directed to a method for synthesizing a cyclic depsipeptide which comprises:
(a) coupling a protected tri-functional molecule with a plurality of protecting groups onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) cleaving a protecting group from either (i) the protected amino acid or peptide, or (ii) the tri-functional molecule so as to form a deprotected amino acid or peptide, or a deprotected tri-functional molecule coupled on the solid support;
(e) coupling a protected cleavable linker with either (iii) the deprotected amino acid or peptide, or (iv) the deprotected tri-functional molecule;
(f) cleaving the protecting group from the cleavable linker and a protecting group from either (iii) the protected amino acid or peptide, or (iv) the protected tri-functional molecule and cyclizing so as to form a cyclic depsipeptide coupled on the solid support; and
(g) cleaving any remaining protecting groups from the cyclic depsipeptide coupled on the solid support.

In the cyclic depsipeptide embodiment above, a solid-phase library of cyclic depsipeptides may be prepared. It may comprise an additional step (h) wherein the library of cyclic depsipeptides on the solid support is screened to identify a cyclic depsipeptide(s) that bind to a biologic of interest. It may further comprise additional step (h) wherein the ester bond in the cyclic depsipeptide is hydrolyzed so as to yield a linear molecule on the solid support. It may further comprise additional step (h) wherein the cyclic depsipeptide is cleaved from the solid support.

Alternatively, it may further comprise additional step (h) wherein both the cyclic depsipeptide is cleaved from the solid support, and the ester bond in the cyclic depsipeptide may be hydrolyzed, to yield a linear molecule. The linear molecule may be sequenced by Edman degredation or mass spectrometry.

The cleavable linker may be either an ester forming or an ester containing cleavable linker. The ester forming cleavable linker may be either a hydroxyl protected or a hydroxyl unprotected linker, or a monoprotected dicarboxylic acid linker. The hydroxyl protected ester forming cleavable linker may be an N,O-protected hydroxy amino acid. The hydroxyl unprotected ester forming cleavable linker may be a carboxyl-protected α-, β-, or γ-hydroxyacid.

The ester forming cleavable linker may be a mono-ester of a dicarboxylic acid, carboxyl-protected α-hydroxy acid, a lactic acid ester, an alkyl lactate or an alkenyl lactate.

The ester containing cleavable linker may be either the ester of an Nα-protected amino acid and a hydroxy acid, or the ester of an amino acid and a carboxyl-protected hydroxy acid, or the ester of an Nα-protected amino acid and an Nα-acylated hydroxy amino acid.

In yet another embodiment, the invention is directed to a method for synthesizing a cyclic amidine-peptide, the method comprising:
(a) coupling a protected tri-functional molecule onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) deprotecting a primary amino group from the protected amino acid or peptide and a primary amino group from the tri-functional molecule so as to form a deprotected amino group on the acid or peptide and a deprotected amino group on the tri-functional molecule coupled on the solid support;
(e) reacting a bis-imidoester linker with the primary amino group on the acid or peptide and the primary amino group on the tri-functional molecule coupled on the solid support so as to form an cyclic amidine-peptide coupled on the solid support; and
(f) cleaving any remaining protecting groups from the cyclic amidine-peptide coupled on the solid support.

In the cyclic amidine-peptide embodiment above, a solid-phase library of cyclic amidine-peptides may be prepared. It may comprise an additional step (g) wherein the library of cyclic amidine-peptides on the solid support is screened to identify a cyclic amidine-peptide(s) that bind to a biologic of interest. It may further comprise additional step (g) wherein the ester bond in the cyclic amidine-peptide is hydrolyzed so as to yield a linear molecule on the solid support. It may further comprise additional step (g) wherein the cyclic amidine-peptide is cleaved from the solid support.

Alternatively, it may further comprise additional step (g) wherein both the cyclic amidine-peptide is cleaved from the solid support, and the ester bond in the cyclic amidine-peptide may be hydrolyzed, to yield a linear molecule. The linear molecule may be sequenced by Edman degredation or mass spectrometry. The imidoester linker may be either a homobifunctional or a heterobifunctional imidoester linker.

For either the cyclic depsipeptide or amidine-peptide embodiments above, the linked protected amino acid or peptide in step (c) is reacted in suitable conditions so as to add a plurality of protected amino acids to the linked amino acid or peptide on the partially deprotected tri-functional molecule.

A method of solid-phase synthesis of a cyclic homodetic peptide that binds a biologic of interest which comprises synthesizing a plurality of cyclic depsipeptide or amidine-peptide by the methods above and further comprises additional steps: selecting a cyclic depsipeptide or amidine-peptide that binds to a biologic of interest; sequencing the selected cyclic depsipeptide or amidine-peptide; and synthesizing a cyclic homodetic peptide with a sequence corresponding to the depsipeptide or amidine-peptide.

The invention is also directed to a solid-phase library of cyclic depsipeptides or cyclic amidine-linked peptides wherein each depsipeptide or cyclic amidine-linked peptide independently has the structure:

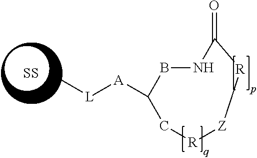

a. A, B, and C, are independently C₁₋₈ alkyl, C₂₋₈ alkenyl, C₃₋₈ alkynyl, or C₁₋₈ alkoxy;
b. p and q are independently integers 0-30 with the proviso that the sum of p and q is greater than 2;
c. each R is independently a biomonomer;
d. L is a suitable linker to the solid support;
e. Z is an ester bond [—CO—O—] or [—O—CO—]; or —(CH₂)_{y}(NH(Ac))(CH₂)ₓCO—O—, —(CH₂)_{y}(NHCO)(CH₂)ₓCO—O—, —(CH₂)_{y}(H(Ac))(CH₂)ₓ)OC—O—, —(CH₂)_{y}(NHCO)(CH₂)ₓOC—O—, —CO—O—(CH₂)ₓ(NH(Ac))(CH₂)_{y}—, —CO—O(CH₂)ₓ(NHCO)(CH₂)_{y}—, —OCO(CH₂)ₓ(NH(Ac))(CH₂)_{y}—, —OCO(CH₂)ₓ(NHCO)(CH₂)_{y}—; or an amidine bond —C(═NH)—NH— or —NHC(═NH);
   —(CH₂)_{y}(NH(Ac))(CH₂)ₓC(═NH)—NH—, —(CH₂)_{y}(NHCO)(CH₂)ₓC(═NH)—NH, —(CH₂)_{y}(NH(Ac))(CH₂)ₓNHC(═NH)—, —(CH₂)_{y}(NHCO)(CH₂)ₓNHC(═NH), —C(═NH)—NH(CH₂)ₓ(NH(Ac))(CH₂)_{y}—, —C(═NH)—NH(CH₂)ₓ(NHCO)(CH₂)_{y}—, —NHC(═NH)(CH₂)ₓ(NH(Ac))(CH₂)_{y}—, —NHC(═NH)(CH₂)ₓ(NHCO)(CH₂)_{y}—; and
f. x is an integer from 1-8 and y is an integer from 0-8.

In one embodiment of the library, the sum of p and q is 2-20, alternatively sum of p and q is 4-10.

### 5.1. DEFINITIONS

The term “biological” includes biopharmaceuticals or biotherapeutics, such as therapeutic proteins. These may be protein therapeutics with enzymatic and/or regulatory activity; or proteins with special binding activity, such as monoclonal antibodies or Fc-fusion proteins; or protein vaccines; or diagnostic proteins. Biologicals may be isolated from living organisms, such as blood factors, or produced by recombinant technology. See Strohl and Knight, Curr Opin Biotech, (2009) 20:668-672, the contents of which are hereby incorporated by reference in its entirety. As used herein, biological also includes viruses and microorganisms such as bacteria, fungi, unicellular or multicellular organisms. In some non-limiting embodiments, a biological may be a pathogenic protein such as a prion, or a pathogenic microorganism such as bacteria, e.g., tuberculosis or anthrax; fungi, e.g., Candida albicans; protozoa, e.g., Plasmodium falciparum; or a multicellular parasite such as Schistosoma mansoni.

A “biopolymer” is a polymer of one or more types of repeating units. Biopolymers can be found in natural biological systems and particularly include oligosaccharides and polysaccharides, peptides (which term is used to include polypeptides and proteins), and polynucleotides (which term is used to include DNA and RNA), or can be produced by artificial biosynthesis, such as peptoids and peptide nucleic acids (PNA). As used herein, the term “biopolymer” includes synthetic compounds having biological activity, such as analogs of naturally occurring compounds composed of or containing amino acids or amino acid analogs, sugars or sugar analogs, or nucleotides or non-nucleotide groups.

The term “biomonomer” means and includes a single unit, which can be linked with the same or other biomonomers to form a biopolymer; for example, an amino acid, a nucleotide, or a saccharide, having one or more linking groups which may have removable protecting groups). Biomonomers may also include compounds such as spacers, for example diamines or dicarboxylic acids, and mixtures thereof, on a hydrocarbon or polyether tether, for example aminoalkanoic acids.

Biomonomers or biopolymers of the current invention may be protected with one or more “protecting groups” that mask the reactivity of functional groups to prevent unwanted side reactions and that can be cleanly removed at a later synthetic stage. See Isidro-Llobet et al., Amino Acid-Protecting Groups, Chem Rev 2009, 109 2455-2504, the contents of which are incorporated by reference in its entirety. Non-limiting examples of protecting groups include:

Alkaline-Stable Amino Protecting Groups:

2-(2-Nitrophenyl)propyloxycarbonyl (NPPOC), 2-(3,4-Methylenedioxy-6-nitrophenyl)propyloxycarbonyl (MNPPOC), 2-(4-Biphenyl)isopropoxycarbonyl (Bpoc), 2,2,2-Trichloroethyloxycarbonyl (Troc), 2,4-Dinitrobenzenesulfonyl (dNBS), 2-Chlorobenzyloxycarbonyl (Cl—Z), 2-Nitrophenylsulfenyl (Nps), 4-Methyltrityl (Mtt), 9-(4-Bromophenyl)-9-fluorenyl (BrPhF), Allyloxycarbonyl (Alloc), Azidomethoxycarbonyl (Azoc), Benzyloxycarbonyl (Z), o-Nitrobenzyloxycarbonyl (oNZ) and 6-Nitroveratryloxycarbonyl (NVOC), p-Nitrobenzyloxycarbonyl (pNZ), Propargyloxycarbonyl (Poc), tert-Butyloxycarbonyl (Boc), Trityl (Trt), α,α-Dimethyl-3,5-dimethoxybenzyloxycarbonyl (Ddz), and α-Azido Carboxylic Acids.

Alkaline-Labile Amino Protecting Groups:

(1-(4,4-Dimethyl-2,6-dioxocyclohex-1-ylidene)-3-ethyl) (Dde), (1,1-Dioxobenzo[b]thiophene-2-yl)methyloxycarbonyl (Bsmoc), (1,1-Dioxonaphtho[1,2-b]thiophene-2-yl)methyloxycarbonyl (r-Nsmoc), 1-(4,4-Dimethyl-2,6-dioxocyclohex-1-ylidene)-3-methylbutyl (ivDde), 2-(4-Nitrophenylsulfonyl)ethoxycarbonyl (Nsc), 2-(4-Sulfophenylsulfonyl)ethoxycarbonyl (Sps), 2,7-Di-tert-butyl-Fmoc (Fmoc*), 2-[Phenyl(methyl)sulfonio]ethyloxycarbonyl tetrafluoroborate (Pms), 2-Fluoro-Fmoc (Fmoc(2F)), 2-Monoisooctyl-Fmoc (mio-Fmoc) and 2,7-Diisooctyl-Fmoc (dio-Fmoc), 9-Fluorenylmethoxycarbonyl (Fmoc), Ethanesulfonylethoxycarbonyl (Esc), and Tetrachlorophthaloyl (TCP).

Alkaline-Stable Carboxylic Acid Protecting Groups:

(2-Phenyl-2-trimethylsiylyl)ethyl (PTMSE), 1,1-Dimethylallyl (Dma), 2-(Trimethylsilyl)isopropyl (Tmsi), 2,2,2-Trichloroethyl (Tce), 2,4-Dimethoxybenzyl (Dmb), 2-Chlorotrityl (2-Cl-Trt), 2-Phenylisopropyl (2-PhiPr), 2-Phenylisopropyl (2-PhiPr), 2-Trimethylsilylethyl (TMSE), 4-(3,6,9-Trioxadecyl)oxybenzyl (TEGBz or TEGBn), 4,5-Dimethoxy-2-nitrobenzyl (Dmnb), 5-Phenyl-3,4-ethylenedioxythenyl Derivatives (Phenyl-EDOTn), Allyl (Al), Benzyl (Bn), Cyclohexyl (cHx), Pentaamine Cobalt(III), Phenacyl (Pac), p-Hydroxyphenacyl (pHP), p-Nitrobenzyl (pNB), tert-Butyl (tBu), β-3-Methylpent-3-yl (Mpe), and β-Menthyl (Men).

Alkaline-Labile Carboxylic Acid Protecting Groups:

9-Fluorenylmethyl (Fm), Methyl (Me) and Ethyl (Et), Carbamoylmethyl (Cam), and 4-(N-[1-(4,4-dimethyl-2,6-dioxocyclohexylidene)-3-methylbutyl]amino)benzyl (Dmab),

Thiol Protecting Groups:

2,2,4,6,7-Pentamethyl-5-dihydrobenzofuranylmethyl (Pmbf), 2-Pyridinesulfenyl (S-Pyr), 3-Nitro-2-pyridinesulfenyl (Npys), 4-Picolyl, 9-Xanthenyl (Xan), Acetamidomethyl (Acm), Allyloxycarbonyl (Alloc), Benzyl (Bn), Monomethoxytrityl (Mmt), N-Allyloxycarbonyl-N-[2,3,5,6-tetrafluoro-4-(phenylthio), o-Nitrobenzyl (oNB), phenyl]aminomethyl (Fsam), Phenylacetamidomethyl (PhAcm), p-Methoxybenzyl (Mob), p-Methylbenzyl (Meb), tert-Butyl (tBu) and 1-Adamantyl (1-Ada), tert-Butylmercapto (StBu), Trimethoxybenzyl (Tmob), and Trityl (Trt).

The term “solid support” as used herein refers to hydrophilic porous materials. Also, porous material with a hydrophilic coating may be used in the present invention. Solid supports include inorganic materials, organic materials, and combinations thereof. It may be a hydroxylated or aminated solid support or a hydroxylated or aminated composite solid support. The solid support may be a polyacrylamide or a derivative thereof, polyacrylate and a derivative thereof, hydrophilically coated polystyrene, or poly(ethylene glycol). The solid support material may be in the form of porous beads, which may be spherical. Alternatively, the support may be porous particulate or divided form having other regular or irregular shapes. Other examples of suitable solid support materials include membranes, capillaries, microarrays, monolites, multiple-well plates. Solid supports of the present invention may be rigid or non-rigid flexible materials such as a fabric which may be woven or non-woven.

In one embodiment, preferred solid support materials are those having minimal non-specific protein binding properties and that are physically and chemically resistant to the conditions used for organic synthesis as well as for the purification process employed in this invention that might involve changes in pH and ionic strength in aqueous environment. The solid support material may be poly(ethylene glycol) based, such as a PEG-based hydrophilic resin like ChemMatrix®. Another solid support used in the present invention may be polyacrylate or a derivative thereof. Examples of acrylate polymers include, but are not limited to, poly(methacrylate), poly(hydroxy methacrylate), poly(methyl methacrylate), polyacrylamide, polyacrylonitrile and other acrylate derivatives.

### 5.2. SYNTHESIS OF CYCLIC DEPSIPEPTIDES AND LIBRARIES THEREOF

This section presents a method for the solid-phase synthesis of cyclic depsipeptides and libraries thereof. The main components of the proposed molecules are the “key stone” tri-functional molecule, upon which the cyclic depsipeptide is constructed, and a cleavable linker, which allows the ring opening in a known position to return the depsipeptide to its linear structure.

Without limiting the scope of the invention, this disclosure presents the depsipeptide cyclization as head-to-side chain or side chain-to-side chain reaction. Furthermore, in the method presented, the treatment used to open the ring can also release the peptide from the solid support to the liquid phase, making it available for analysis in liquid phase.

Section 5.2.1 lists some of the Y-shaped protected tri-functional molecules upon which the cyclic depsipeptides are articulated; section 5.2.2 lists some of the cleavable linkers and their uncleavable analogues that can be used in some of the embodiments; section 5.2.3 presents general methods for the synthesis of cyclic depsipeptides and libraries thereof using the listed linkers as well as the methods for the synthesis of the corresponding peptide analogues; section 5.2.4 presents detailed protocols for the synthesis of example sequences.

5.2.1. “Key Stone” Protected Tri Functional Molecules for the Synthesis of Cyclic Depsipeptides: Amino Dicarboxylic Acid and Diamino Acids

Amino Dicarboxylic Acid

Without limiting the scope of the invention, this disclosure presents the use of protected aspartic acid and glutamic acid as examples of amino dicarboxylic acids to be used as “key stone” protected tri-functional molecules. Without limiting the scope of the invention, protecting group for the Nα group can be Fmoc and tBoc, while protecting group for the γ, δ-carboxyl group on respectively aspartic acid and glutamic acid can be methyl ester (OMe), ethyl ester (OEt), allyl ester (OAll), p-nitrobenzyl ester (pNb), and others. Some examples are shown in FIG. 2.

The conditions for cleaving the above mentioned group are: for methyl and ethyl esters, LiI in pyridine or LiI in ethyl acetate; for allyl ester, 0.1 eq of Pd(PPh₃)₄ in DCM and 10 eq. PhSiH₃ as scavenger; for p-nitrobenzyl ester, 6 eq. of SnCl₂ in DMF and 0.16 eq of HCl in dioxane; for Fmoc, 20% Piperidine in DMF; for tBoc: 50% TFA in DCM.

Diamino Acid

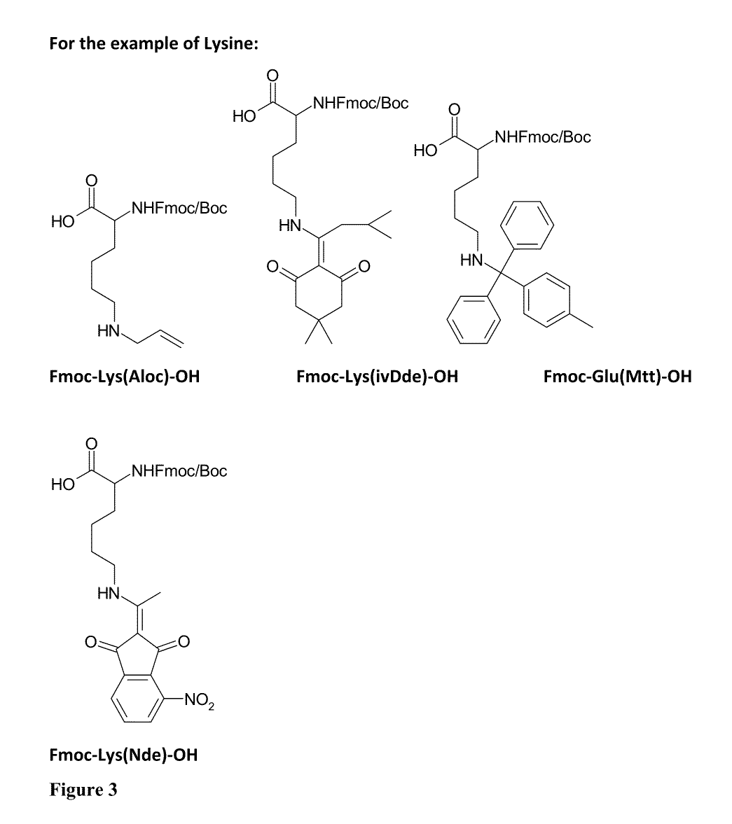

Without limiting the scope of the invention, this disclosure also presents the use of protected lysine, ornithine, diamino butanoic acid, and diamino propionic acid as examples of diamino acids to be used as “key stone” protected tri-functional molecules. Without limiting the scope of the invention, protecting group for the Nα group can be Fmoc and tBoc, while protecting group for the β, γ, δ, ε-amino group on the side chain can be allyloxycarbonyl (Aloc), Dde/ivDde, Mtt, and Nde. Some examples are shown in FIG. 3.

The conditions for cleaving the above mentioned group are: for Aloc, 0.1 eq of Pd(PPh₃)₄ in DCM and 10 eq. PhSiH₃ as scavenger; for Dde and ivDde, 2% hydrazine in DMF; for Mtt, 1% TFA in DCM; for Nde, 2% hydrazine in DMF; for Fmoc, 20% Piperidine in DMF; for tBoc: 50% TFA in DCM.

5.2.2. Cleavable Linkers for the Synthesis of Cyclic Depsipeptides

The cleavable linkers presented in this section can be either ester forming or ester containing. The former are hetero multifunctional monomers, either hydroxyl-protected or hydroxyl-unprotected, that can be true amino acids or structurally similar to amino acids, such as hydroxy acids. The latter are heterobifunctional compounds, either protected or unprotected, that contain an ester bond in their structure. Examples of these cleavable linkers are reported below.

5.2.2.1. Ester Forming Cleavable Linkers

Ester forming cleavable linkers can be either (i) hydroxyl-protected or (ii) hydroxyl-unprotected or (iii) carboxyl-protected.

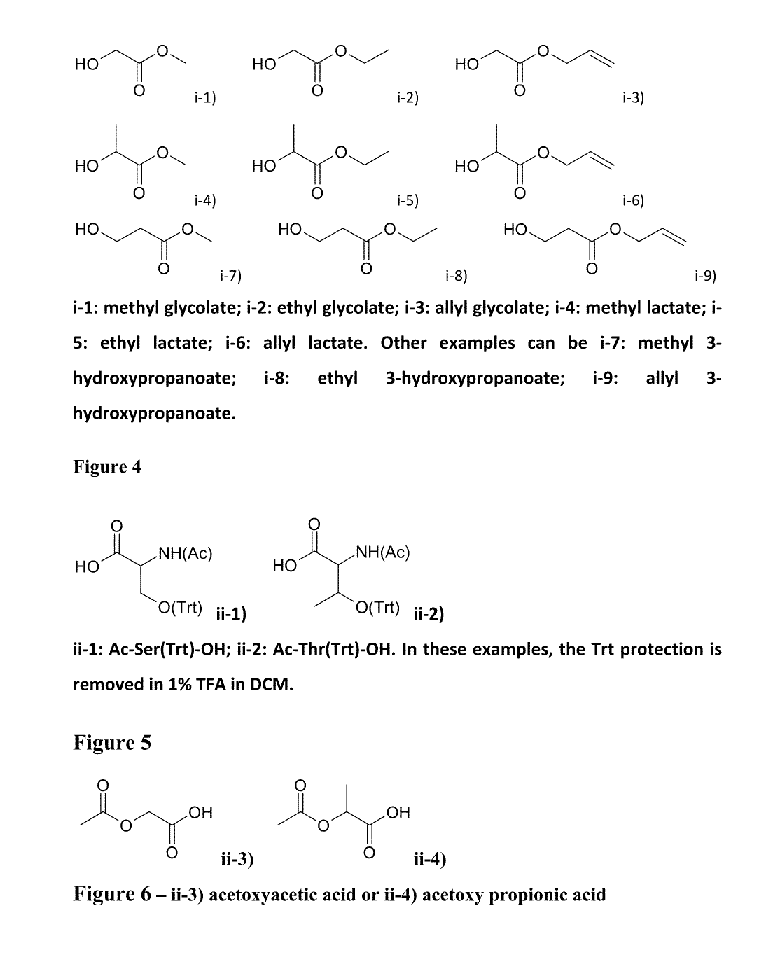

Hydroxyl-unprotected ester forming cleavable linkers can be carboxyl-protected α-, β-, or γ-hydroxy acids (FIG. 4).

Hydroxyl-protected ester forming cleavable linkers can be N,O-protected hydroxy amino acids, such as serine and threonine or hydroxy-analogue amino acids (FIG. 5).

A further example of hydroxyl-protected ester forming cleavable linker can be acetoxyacetic acid or acetoxy propionic acid (FIG. 6). The hydroxyl-protection is cleaved with hydrazine monohydrate in dimethylacetamide (DMA).

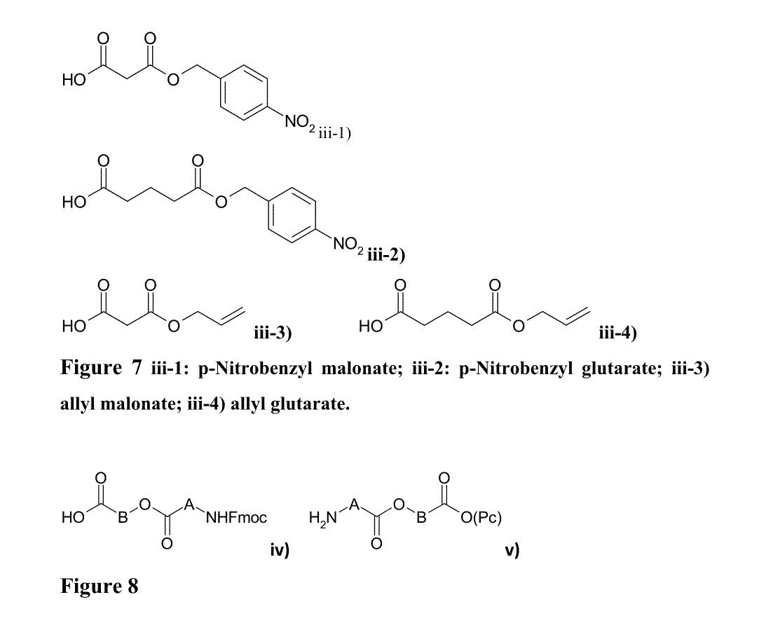

Carboxyl-protected ester forming cleavable linkers can be mono-protected dicarboxylic acid. Examples are presented in FIG. 7.

5.2.2.2. Ester Containing Cleavable Linkers

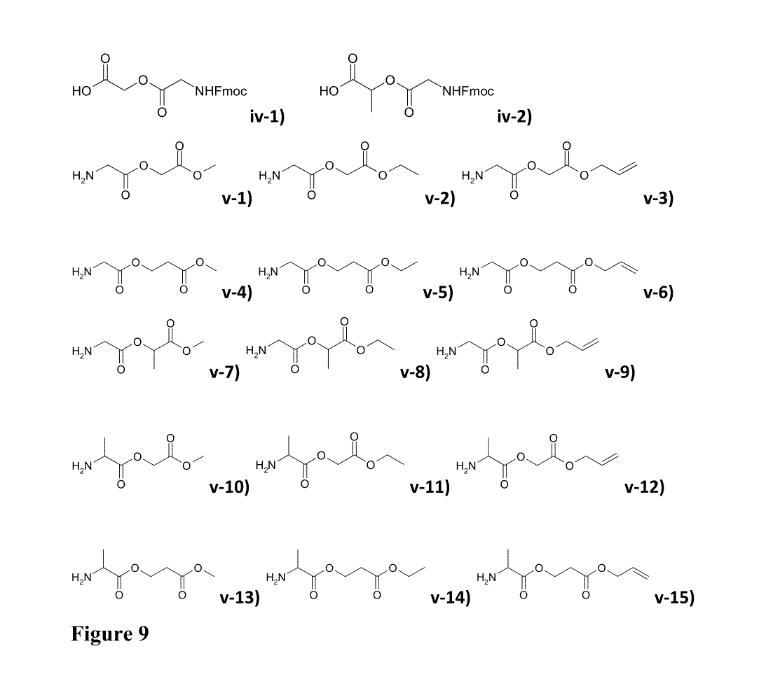

Without limiting the scope of the invention, this disclosure provides two main classes of ester containing heterobifunctional cleavable linkers (FIG. 8): iv) the ester of an N-protected amino acid and a hydroxy acid and v) the ester of an amino acid and a C-protected hydroxy acid. Examples are presented in FIG. 9.

5.2.3. General Methods for the Synthesis of Cyclic Depsipeptides and Libraries Thereof

In this section of the disclosure, several methods/embodiments are presented for the synthesis of cyclic depsipeptides, also called herein reversible cyclic peptides, and libraries thereof.

Without wishing to limit the scope of this invention, two resins are used herein for solid-phase synthesis of depsipeptides, i.e. a poly(ethylene glycol) based aminoethyl ChemMatrix® (PCAS BioMatrix Inc., Quebec, Canada) and hydroxyl resin and a poly(methacrylate) based Toyopearl amino resin (Tosoh Bioscience, PA, USA). These resins were selected as they are both stable to the conditions employed for peptide synthesis, such as repeated rinses with organic solvents and contact with organic reagents and strong organic acids, as well as to the alkaline treatment employed for linearization and, when needed, cleavage of the depsipeptide. In addition, both resin present low non-specific binding of proteins and high functional density. Camperi S. A., Marani M. M., Iannucci N. B., Cote S., Albericio F. and Cascone O. (2005), An efficient strategy for the preparation of one-bead-one-peptide libraries on a new biocompatible solid support, Tetrahed. Lett. 46, 1561-1564; Martinez-Ceron M. C., Giudicessi S. L., Marani M. M., Albericio F., Cascone O. Erra-Balsells R. and Camperi S. (2010), Sample preparation for sequencing hits from one-bead-one-peptide combinatorial libraries by matrix-assisted laser desorption/ionization time-of-flight mass spectrometry, Anal. Biochem. 400, 295-297; Marani M. M., Oliveira E., Cote S., Camperi S. A., Albericio F. and Cascone O. (2007), Identification of protein-binding peptides by direct matrix-assisted lased desorption ionization time-of-flight mass spectrometry analysis of peptide beads selected from the screening of one-bead-one-peptide combinatorial libraries, Anal. Biochem. 370, 215-222. While ChemMatrix is highly suitable to peptide synthesis but it lacks of the mechanical stability required to a chromatographic resin, it is employed for synthesis and screening of the depsipeptide library. Toyopearl amino, being highly mechanically stable, is employed for the following chromatographic characterization of the selected sequences.

Furthermore, in the following procedures specific protected amino acids and linkers are indicated for the synthesis of cyclic depsipeptides and their corresponding cyclic peptide analogues. These choices are just meant for clarification and as exampled and not to limit the scope or the broadness of the invention.

Finally, the following procedures involve the coupling of protected amino acids and linkers on primary amino groups and carboxyl groups. To monitor the coupling efficiency, qualitative tests are provided for both functional groups. Kaiser test is commonly employed for the detection of free amino groups, so that, if positive, it indicates that the coupling reaction onto amino group is not complete. A colorimetric test for detecting the presence of free carboxyl groups has been proposed by Zamir et al. (Anal. Chim. Acta 1955, 12, 577-579).

5.2.4. Method for the Synthesis of Cyclic Depsipeptides and Libraries Thereof Using Dicarboxylic Amino Acid and Hydroxyl-Unprotected Ester Forming Cleavable Linkers

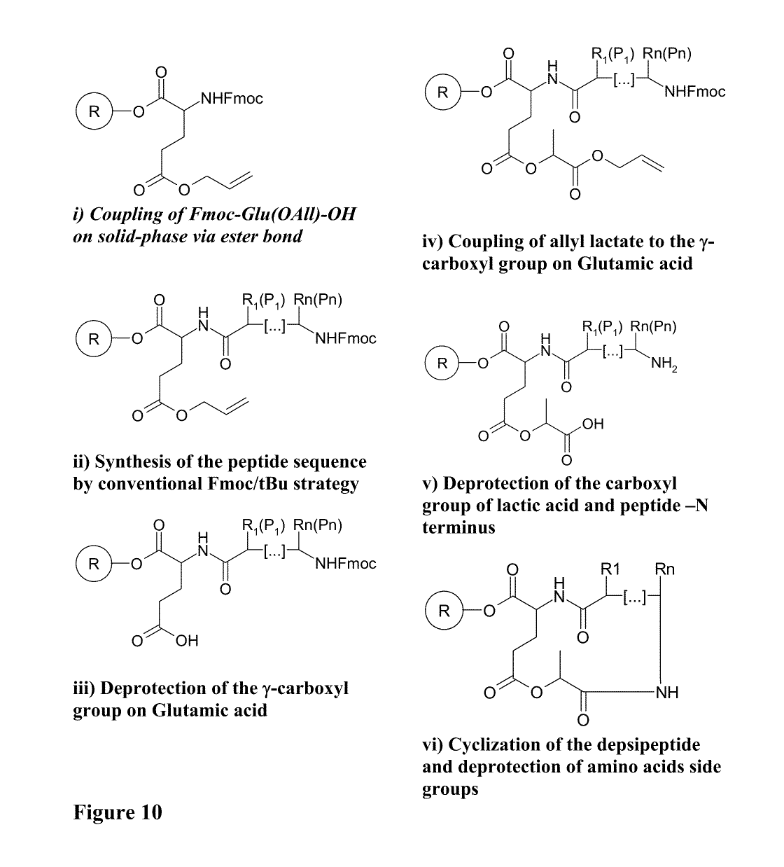

In one embodiment, a dicarboxylic acid as tri-functional molecule and a hydroxyl-unprotected ester forming cleavable linker are adopted for the synthesis of cyclic depsipeptides. (FIG. 10, i) The synthesis begins by coupling of Nα-protected, carboxyl protected dicarboxylic acid, like Fmoc protected glutamic acid allyl ester (Fmoc-Glu(OAll)-OH), onto a 4-hydroxymethylbenzoic acid (HMBA) linker coupled on aminoethyl ChemMatrix®. The allyl ester (OAll) protection on the γ-carboxyl group of glutamic acid is an orthogonal protecting group in the Fmoc/tBu strategy for peptide synthesis. The glutamic acid is coupled to the HMBA linker through an ester bond, which can be cleaved in alkaline conditions to release the peptide in solution. (ii) Peptide synthesis is then performed by conventional Fmoc/tBu strategy. Camino L. A. and Han G. Y. (1972) The 9-Fluoroenylmethyoxycarbonyl amino-protecting group, J. Org. Chem., 37, 3404-3409. Fields, G. B. and Noble R. L. (1990) Solid phase peptide synthesis utilizing 9-fluorenylmethoxycarbonyl amino acids, Int. J. Peptide Protein Res., 84, 643-649. (iii) The ° All protection on glutamic acid is then removed by using tetrakis(triphenyl-phosphine) palladium(0) catalyst, which does not affect the protections on the side chain of the amino acid residues. Llobet A. I., lvarez M. and Albericio F., (2009), Amino Acid-Protecting Groups, Chem. Rev. 109, 2455-2504. The cleavable linker is selected among the hydroxyl-unprotected ester forming cleavable linkers. Viable options for the linker are the allyl glycolate and allyl lactate. (iv) The hydroxyl group of the linker is reacted with the carboxyl group of glutamic acid to form an alkaline-labile ester bond. (v) The allyl ester protection on the linker and the Fmoc protection on the N-terminus of the peptide are sequentially removed using palladium(0) catalyst and 20% piperidine in DMF respectively. (vi) The peptide is cyclized, using HATU coupling agent and finally the side protecting groups of the amino acid residues are removed via acidolysis.

5.2.4.1. Method for the Synthesis of the Cyclic Peptide Analogue

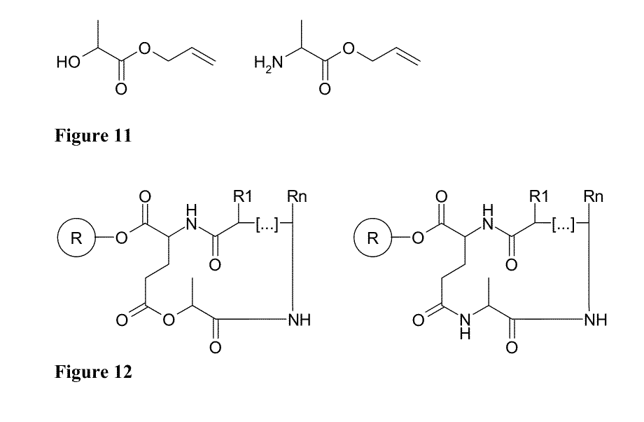

The lead peptide sequences identified after library screening, as is presented in Section 5.5, can be synthesized in a chromatographic format for further screening. In one embodiment, the peptide synthesis may be performed on a chromatographic resin bearing amino groups, such as Toyopearl AF-Amino-650M. The synthesis follows the same steps as described above for the production of cyclic depsipeptides. However, the cleavable linker is to be substituted with a non-cleavable linker. This is to ensure that the peptide retains its cyclic structure, if and when exposed to the alkaline conditions required for the resin cleaning and sanitization. These procedures are routinely employed to ensure the safe resin reusability over a large number of cycles of protein purification. Hober S., Nord K. and Linhult M. (2007) Protein A chromatography for antibody purification. J. Chromatogr. B 848, 40-47. In order to maintain the structure and hence the binding properties of the identified ligand, the uncleavable linker is to be structurally analogue to the cleavable linker. For example, in place of the allyl lactate employed for the synthesis of the cyclic depsipeptide, the structural analogue alanine allyl ester (FIG. 11) can be employed for the synthesis of the corresponding homodetic cyclic peptide (FIG. 12). As FIG. 12 shows, while the cleavable linker is bound to the side chain carboxyl group of glutamic acid through an alkaline-labile ester bond, the uncleavable linker is bound to the same carboxyl group through a stable amide bond.

5.2.5. Method for the Synthesis of Cyclic Depsipeptides and Libraries Thereof Using Dicarboxylic Amino Acid and Hydroxyl-Protected Ester Forming Cleavable Linkers

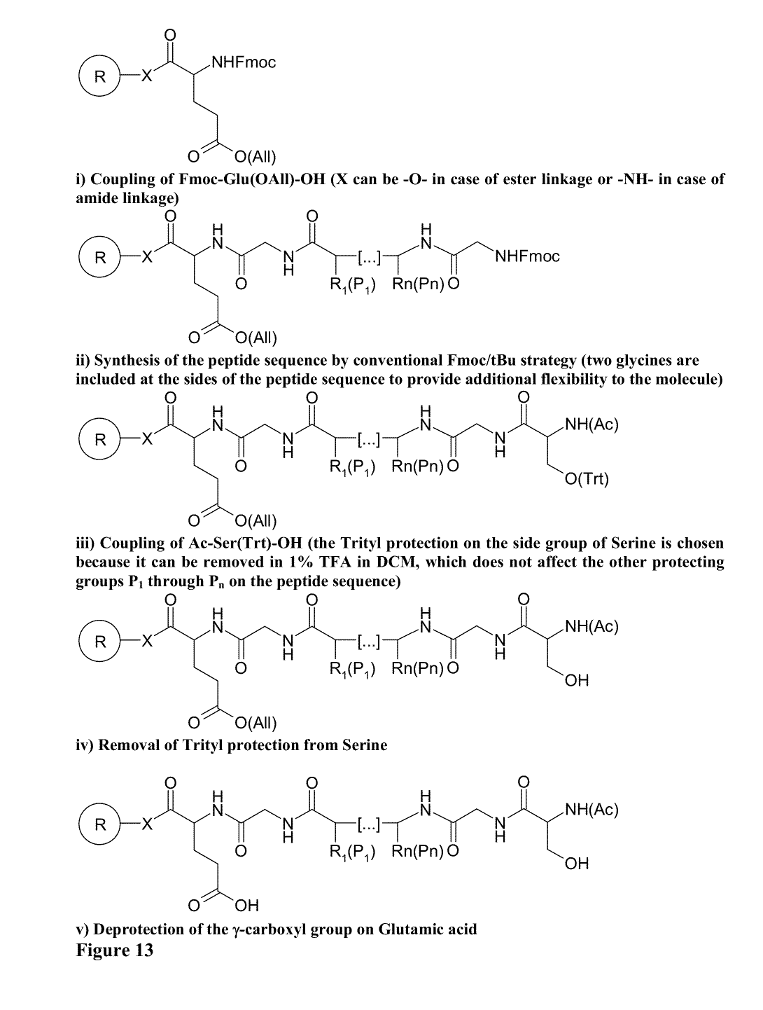

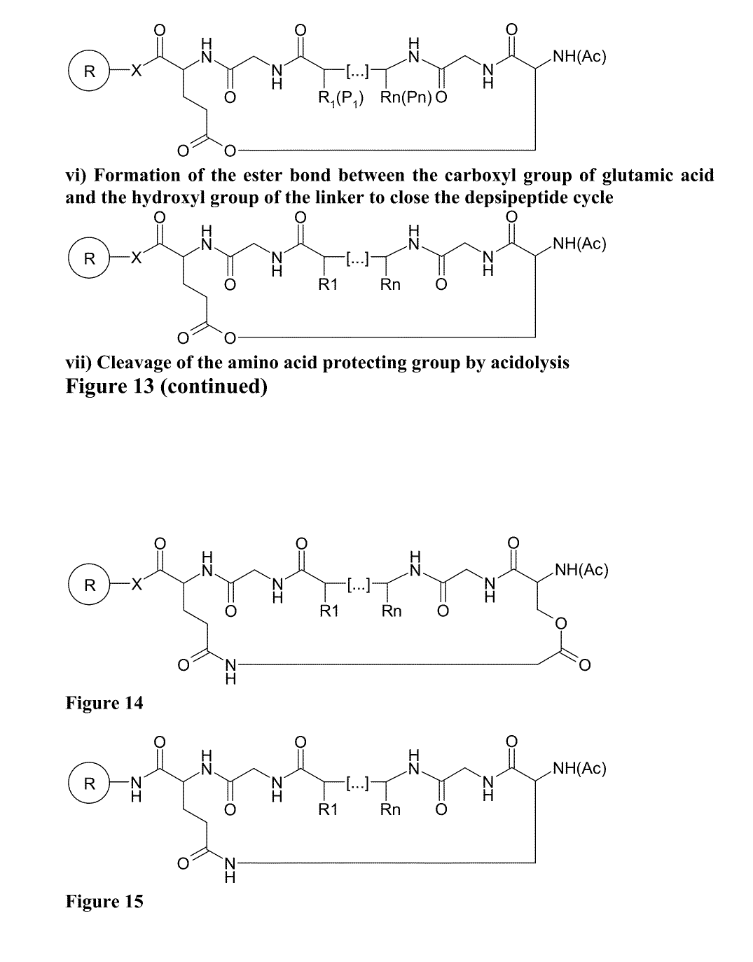

Alternative A: In another embodiment, a dicarboxylic acid as tri-functional molecule and a hydroxyl-protected ester forming cleavable linker are adopted for the synthesis of cyclic depsipeptides. (FIG. 13, i) The synthesis begins by coupling of Fmoc protected glutamic acid allyl ester (Fmoc-Glu(OAll)-OH) onto a 4-hydroxymethylbenzoic acid (HMBA) linker coupled on aminoethyl ChemMatrix®. (ii) Peptide synthesis is then performed by conventional Fmoc/tBu strategy. (iii) The last amino acid in the sequence is N-acylated, O-Trityl protected hydroxyamino acid, such as Ac-Ser(Trt)-OH or Ac-Thr(Trt)-OH. (iv) The Trityl protection on the hydroxyl group of Serine or Threonine is chosen as it can be easily removed in 1% TFA in DCM, leaving the other protecting groups intact. (v) The OAll protection on glutamic acid is then removed by using tetrakis(triphenyl-phosphine) palladium(0) catalyst. (vi) The carboxyl group of glutamic acid is reacted with the hydroxyl group of the linker using HATU coupling agent to form an ester tether so to close the depsipeptide cycle. (vii) Finally the side protecting groups of the amino acid residues are removed by acidolysis.

Alternative B: In analogy, prior to depsipeptide cyclization, an additional Nα-protected amino acid can be coupled by ester bond on the —OH group of the hydroxy amino acid. Fmoc-Gly-OH or Fmoc-Ala-OH are valid alternatives. After cleavage of the OAll protection on glutamic acid and of the Nα-protecting group, the depsipeptide is cyclized by formation of amide bond between the free carboxyl group of glutamic acid and the amino group of the additional amino acid. The resulting structure is presented in FIG. 14.

After linearization, the peptide can be sequenced via MS/MS.

5.2.5.1. Method for the Synthesis of the Cyclic Peptide Analogue

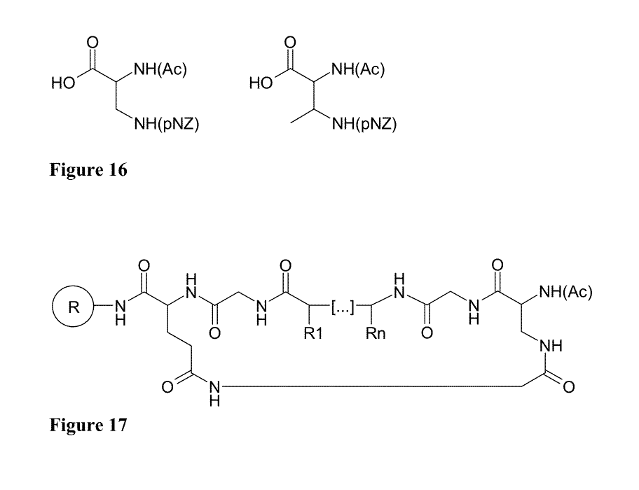

Alternative A: The synthesis follows the same steps as described above for the production of a cyclic depsipeptide with hydroxyl-protected ester forming cleavable linker. The cleavable linker, however, is replaced with a non-cleavable linker. In place of Nα-acylated, O-Trityl protected hydroxy amino acid, a Nα-acylated Nβ-protected diamino acid can be used for the synthesis of the corresponding homodetic cyclic peptide (FIG. 15). In place of Serine, for example, Ac-Dap(Aloc)-OH or Ac-Dap(pNZ)—OH or the like, can be used. In place of Threonine, for example Nα-Ac—Nβ-Aloc-2,3-diaminobutanoic acid or the like can be used (FIG. 16). While the structure is the same, the ester bond is replaced by an alkaline stable amide bond.

Alternative B: With regard to the proposed alternative, the synthesis of the homodetic cyclic peptide (FIG. 17) follows the same procedure. In place of Nα-acylated, O-Trityl protected hydroxy amino acid, the above mentioned Nα-acylated Nβ-protected diamino acid can be used. After cleavage of the Nβ-protecting group, the additional Nα-protected amino acid is be coupled by ester bond on the β-amino group of the diamino acid. After cleavage of the OAll protection on glutamic acid and of the Nα-protecting group, the depsipeptide is cyclized by formation of amide bond between the free carboxyl group of glutamic acid and the amino group of the additional amino acid. Finally, the side protecting groups of the amino acid residues are removed by acidolysis.

5.2.6. Method for the Synthesis of Cyclic Depsipeptides and Libraries Thereof Using Diamino Acid and Carboxyl-Protected Ester Forming Cleavable Linkers

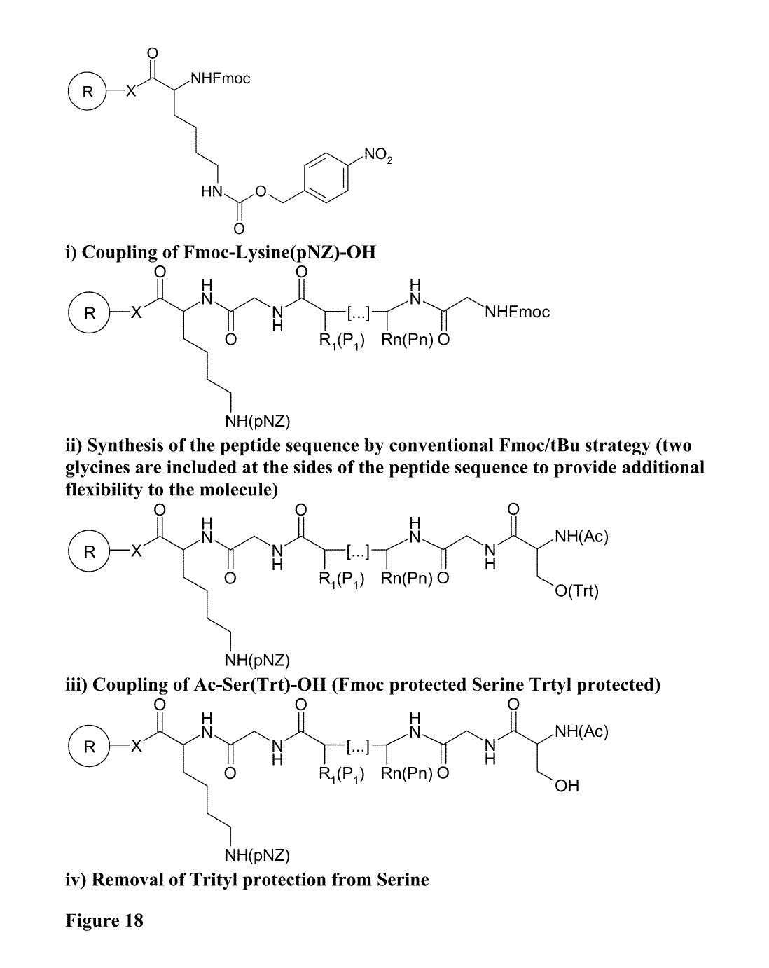

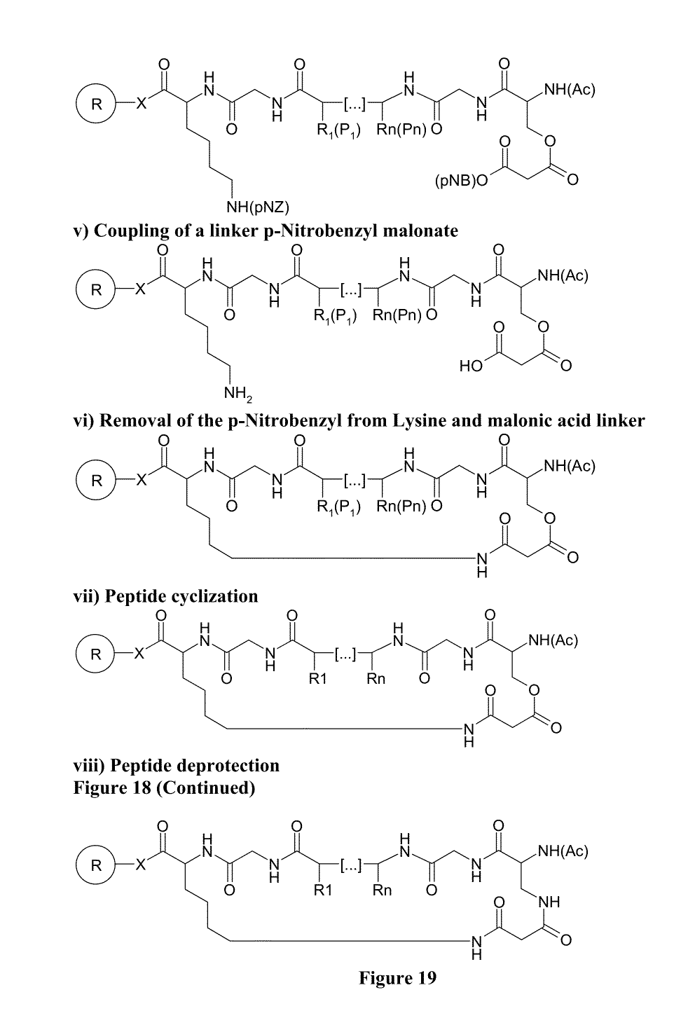

In this embodiment, a diamino acid as tri-functional molecule and a carboxyl-protected ester forming cleavable linker are adopted for the synthesis of cyclic depsipeptides. (FIG. 18, i) The synthesis begins by coupling of an N,N bis-protected diamino acid, like Nα-Fmoc-Nε-pNZ (p-nitrobenzyloxycarboxyl) lysine onto a 4-hydroxymethylbenzoic acid (HMBA) linker coupled on aminoethyl ChemMatrix®. (ii) Peptide synthesis is then performed by conventional Fmoc/tBu strategy. (iii) The last amino acid in the sequence is N-acylated, O-Trityl protected hydroxyamino acid, such as Ac-Ser(Trt)-OH or Ac-Thr(Trt)-OH. (iv) The Trityl protection on the hydroxyl group of Serine or Threonine is chosen as it can be easily removed in 1% TFA in DCM, leaving the other protecting groups intact. (v) A carboxyl-protected ester forming cleavable linker, such as p-nitrobenzyl malonate, is coupled on the hydroxyl group of the hydroxyamino acid by ester bond. (vi) The pNZ and pNB protecting groups are removed respectively from Lysine and malonic acid using tin(II) chloride in slightly acidic DMF (1.6 mM HCl in dioxane)). (vii) The carboxyl group of the linker is reacted with the ε-amino group of lysine using HATU coupling agent to form so to close the depsipeptide cycle. (viii) Finally the side protecting groups of the amino acid residues are removed in acidic conditions.

After linearization, the peptide can be sequenced via MS.

5.2.6.1. Method for the Synthesis of the Cyclic Peptide Analogue

Alternative A: The synthesis follows the same steps as described above. The sole difference consists in replacing the cleavable linker N-acylated, O-Trityl protected hydroxy amino acid with an uncleavable linker Nα-acylated Nβ-protected diamino acid, for the synthesis of the corresponding homodetic cyclic peptide (FIG. 19). Examples of these amino acids are presented in 5.2.3.2. After coupling the uncleavable linker, the rest of the protocol is the same as described above.

5.2.7. Method for the Synthesis of Cyclic Depsipeptides and Libraries Thereof Using Dicarboxylic Amino Acid and Ester Containing Cleavable Linkers

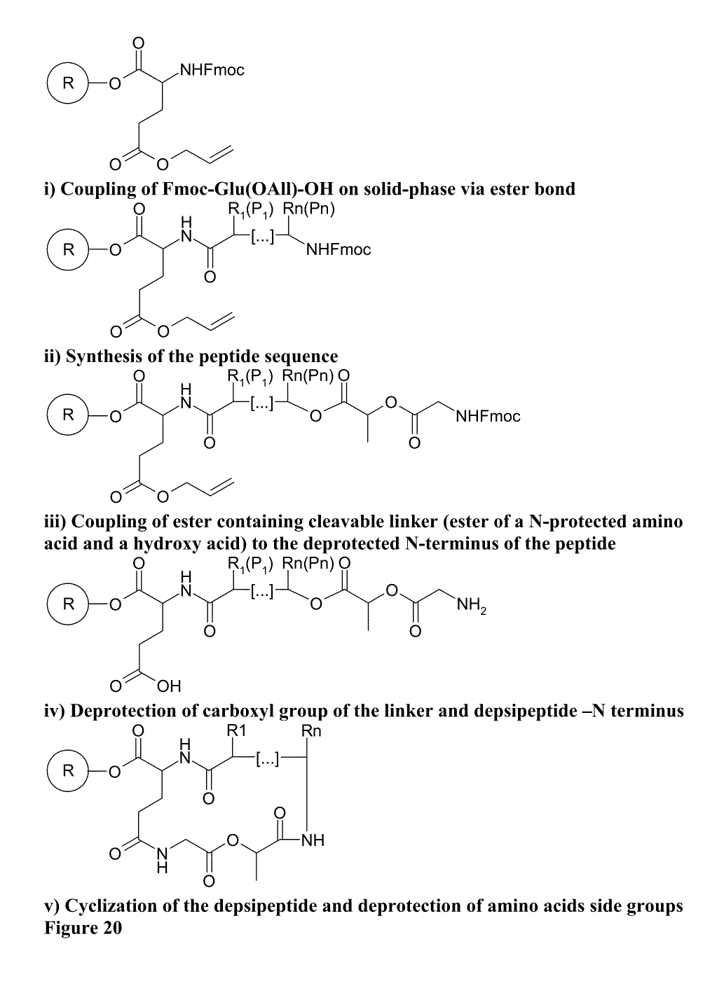

Alternative A: In one embodiment, a dicarboxylic acid as tri-functional molecule and the ester of an N-protected amino acid and a hydroxy acid as ester containing cleavable linker are adopted for the synthesis of cyclic depsipeptides. (FIG. 20, i) The synthesis begins by coupling of Nα-protected, carboxyl protected dicarboxylic acid, like Fmoc-protected glutamic acid allyl ester (Fmoc-Glu(OAll)-OH), onto a 4-hydroxymethylbenzoic acid (HMBA) linker coupled on aminoethyl ChemMatrix®. (ii) Peptide synthesis is then performed by conventional Fmoc/tBu strategy. (iii) The ester containing cleavable linker is coupled on the peptide N-terminus. The cleavable linker is selected among the compounds obtained by ester bond between an N-protected amino acid and a hydroxy acid, for example Fmoc-glycine and lactic acid. (iv) After cleavage of the OAll protection from glutamic acid and of the Fmoc from the cleavable linker, (v) the depsipeptide is cyclized by formation of amide bond between the free carboxyl group of glutamic acid and the free amino group on the linker. Finally, the side protecting groups of the amino acid residues are removed by acidolysis.

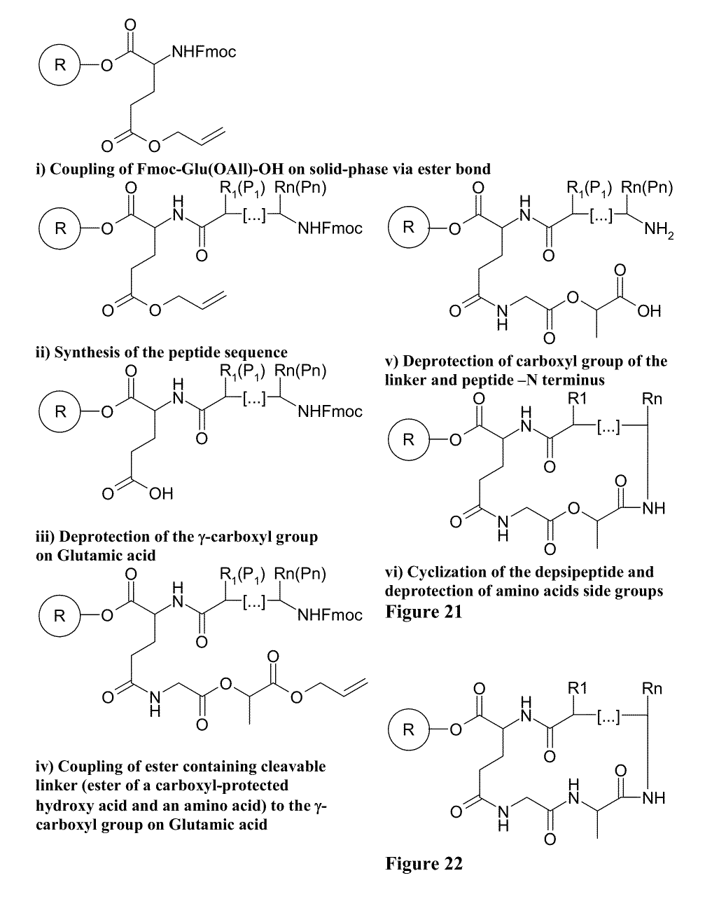

Alternative B: In another embodiment, a dicarboxylic acid as tri-functional molecule and the ester of a carboxyl-protected hydroxy acid and an amino acid as ester containing cleavable linker are adopted for the synthesis of cyclic depsipeptides. (FIG. 21, i) The synthesis begins by coupling of Nα-protected, carboxyl protected dicarboxylic acid, like Fmoc-protected glutamic acid allyl ester (Fmoc-Glu(OAll)-OH), onto a 4-hydroxymethylbenzoic acid (HMBA) linker coupled on aminoethyl ChemMatrix®. (ii) Peptide synthesis is then performed by conventional Fmoc/tBu strategy. The cleavable linker is selected among the cleavable linkers obtained by ester bond between a carboxyl-protected hydroxy acid and an amino acid, for example allyl lactate and glycine. (iii) After cleavage of the OAll protection from glutamic acid, (iv) the free amino group of the linker is reacted with the carboxyl group of glutamic acid to form an alkaline-labile ester bond. (v) The allyl ester protection on the linker and the Fmoc protection on the N-terminus of the peptide are sequentially removed using palladium(0) catalyst and 20% piperidine in DMF respectively. (vi) The peptide is cyclized, using HATU coupling agent and finally the side protecting groups of the amino acid residues are removed via acidolysis.

5.2.7.1. Method for the Synthesis of the Cyclic Peptide Analogue

The synthesis of the homodetic cyclic peptide analogues is performed following the same procedure and by replacing the ester containing linkers with the corresponding amide containing linkers, such as N-protected or C-protected dipeptides (FIG. 22).

5.2.8. Method for the Synthesis of Cyclic Depsipeptides and Libraries Thereof Using a Diamino Acid and Ester Containing Cleavable Linkers

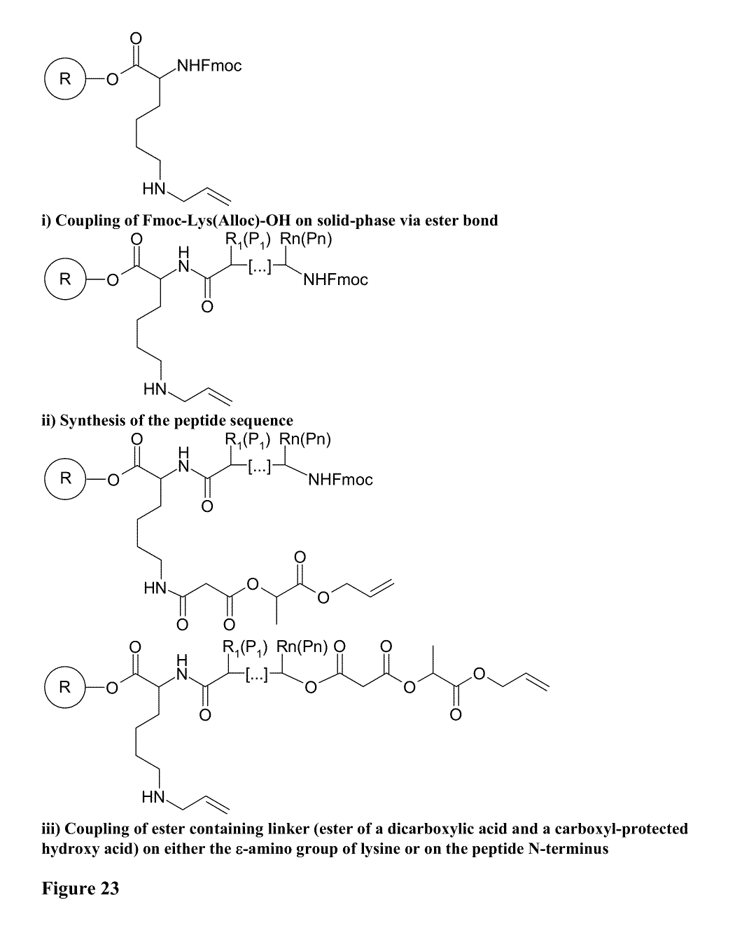

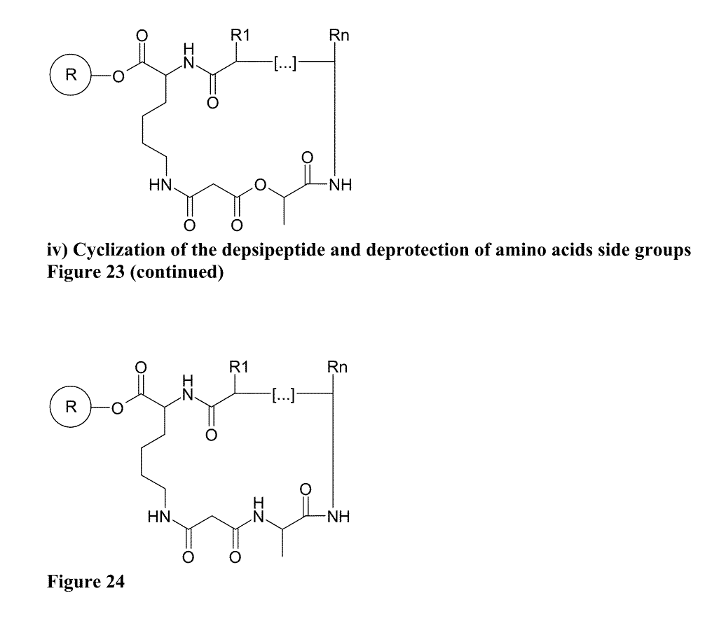

In another embodiment, a diamino acid as tri-functional molecule and the ester of a dicarboxylic acid and a carboxyl-protected hydroxy acid as ester containing cleavable linker are adopted for the synthesis of cyclic depsipeptides. (FIG. 23, i) The synthesis begins by coupling an N,N bis-protected diamino acid, like Nα-Fmoc-Nε-pNZ (p-nitrobenzyloxycarboxyl) Lysine or an Nα-Fmoc-Nε-Aloc Lysine onto a 4-hydroxymethylbenzoic acid (HMBA) linker coupled on aminoethyl ChemMatrix®. (ii) Peptide synthesis is then performed by conventional Fmoc/tBu strategy. The cleavable linker is selected among the compounds obtained by ester bond between a dicarboxylic acid and a carboxyl-protected hydroxy acid, for example malonic acid and allyl lactate. (iii) The linker can be coupled on either the ε-amino group of lysine or on the peptide N-terminus (iv) After deprotection of the linker and the protected amino group, the depsipeptide is cyclized. Finally the side protecting groups of the amino acid residues are removed in acidic conditions.

The synthesis of the homodetic cyclic peptide analogues is performed following the same procedure and by replacing the ester containing linkers with the corresponding amide containing linkers (FIG. 24).

### 5.3. GENERAL METHODS FOR THE SYNTHESIS OF REVERSIBLE CYCLIC AMIDINE-PEPTIDES AND LIBRARIES THEREOF

In this section of the disclosure, several methods are presented for the synthesis of reversible cyclic amidine-peptides and libraries thereof.

Without wishing to limit the scope of this invention, two resins are used herein for solid-phase synthesis of depsipeptides, i.e. a poly(ethylene glycol) based aminoethyl ChemMatrix® (PCAS BioMatrix Inc., Quebec, Canada) and hydroxyl resin and a poly(methacrylate) based Toyopearl amino resin (Tosoh Bioscience, PA, USA).

In this embodiment, the peptide cyclization is performed as a head-to-side chain reaction between two primary amino groups using a homobifunctional imidoester crosslinker. The primary amino groups are made available by diamino acid residues, such as Lysine, Ornithin, Dab, and Dap, in the peptide sequence or by the peptide N-terminus. Each imidoester moiety reacts with a primary amine to form an amidine bond, which can be broken in alkaline conditions or with ammonia. As literature indicates, the imidoester moiety reacts specifically with α-amino groups in a pH range of 8.0-8.5 and also with ε-amino groups at higher pH values, 9.5-10.00. Amidine bonds are stable in acid conditions but are cleaved by aqueous strong alkali or ammonia, to restore the original amino groups. The leads identified from library screening can hence be treated to return the cyclic peptide back to its linear structure and cleave the linear sequence from the resin. The presence of a basic amino acid, such as Lysine, on the C-terminus of the peptide makes the sequence identification particularly suited for ESI-MS/MS. It is in fact well known that a basic amino acid on the C-terminus induces a better fragmentation of the peptides and enhances the quality of the resulting MS spectrum, thereby facilitating the sequence identification. After linearization, the peptide can be sequenced Edman degradation as well because it possesses a free Nα-terminus

In the present embodiment, specific protected amino acids and crosslinkers are indicated for the synthesis of reversible cyclic amidine-peptides and their corresponding cyclic peptide analogues. These choices are just meant for clarification and as exampled and not to limit the scope or the broadness of the invention.

5.3.1. Method for the Synthesis of Reversible Cyclic Amidine-Peptides and Libraries Thereof Using Homobifunctional Imidoester Crosslinkers

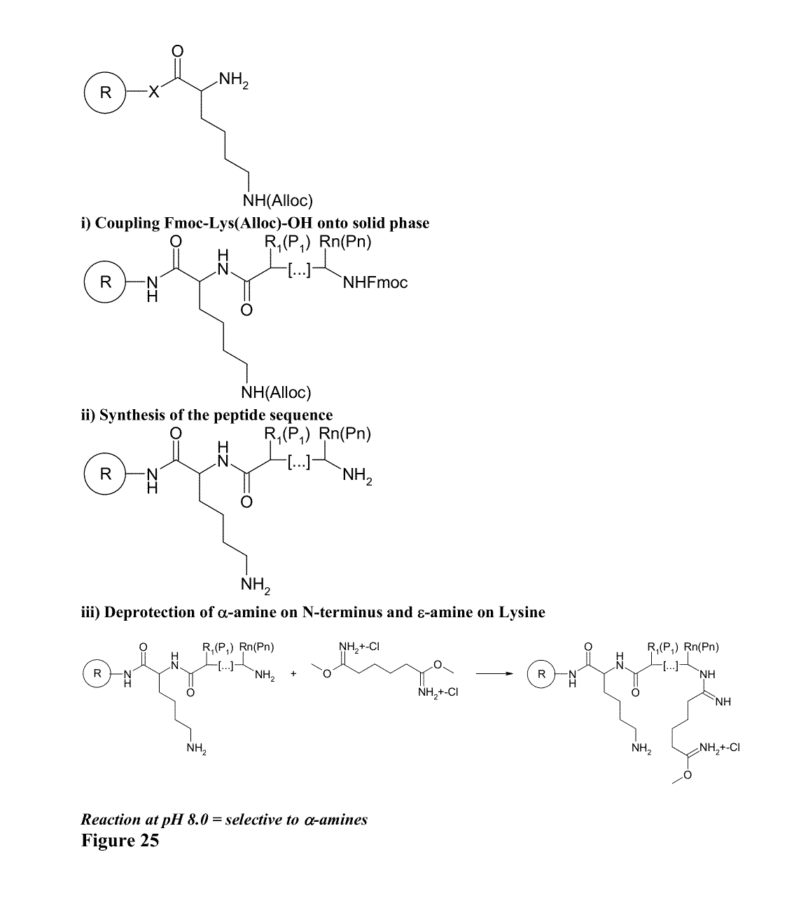

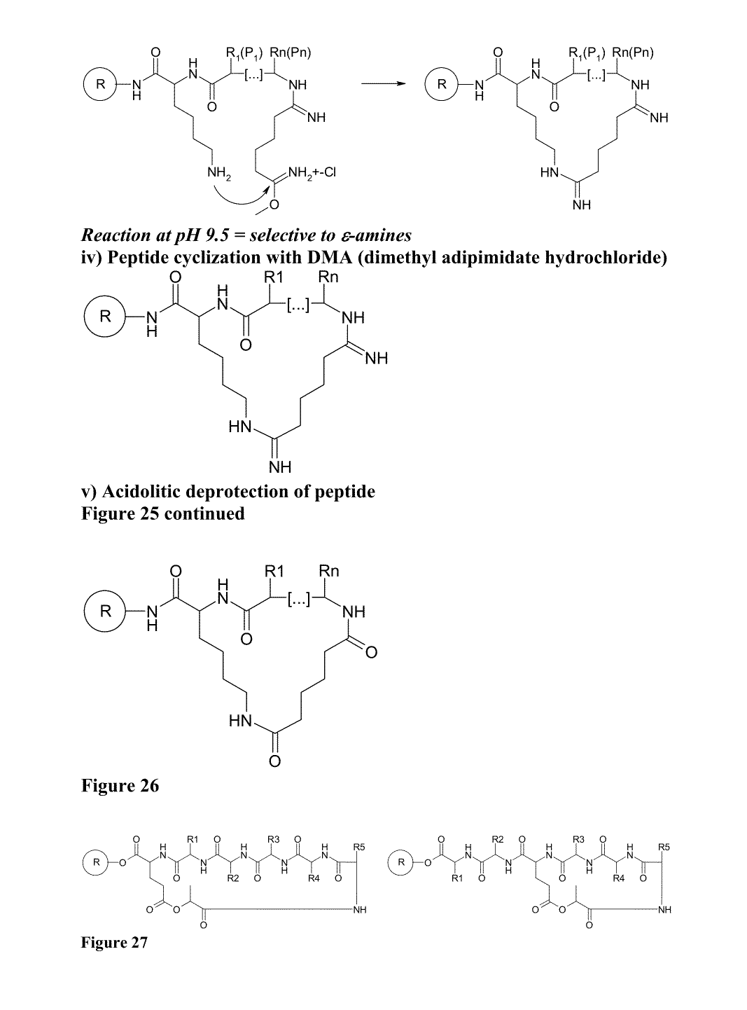

In this embodiment, a diamino acid as tri-functional molecule and a homobifunctional imidoester crosslinker are adopted for the synthesis of reversible cyclic amidine-peptides. (FIG. 25, i) The synthesis begins by coupling of an N,N bis-protected diamino acid, like Nα-Fmoc-Nε-Aloc (allyloxycarbonyl) Lysine onto a 4-hydroxymethylbenzoic acid (HMBA) linker coupled on aminoethyl ChemMatrix®. (ii) Peptide synthesis is then performed by conventional Fmoc/tBu strategy. (iii) The Aloc and Fmoc protecting groups are cleaved respectively from the ε-amino group and the peptide N-terminus using Pd(PPh₃)₄ and 20% piperidine in DMF. A homobifunctional imidoester crosslinker, such as dimethyl adipididate, dimethyl pimelimidate, or dimethyl suberimidate, is used for peptide cyclization by intramolecular crosslinking. (iv) After equilibrating the resin at pH 8.0-8.5 with a suitable coupling buffer, the bis-imidoester is reacted with the ε-amino group of the peptide N-terminus. The resin is then rinsed with the coupling buffer to remove the unreacted crosslinker and to increase the pH to 9.5-10.0. At this new pH value the free imidoester moiety react with the available ε-amino group of Lysine. (v) Finally the side protecting groups of the amino acid residues are removed in acidic conditions.

5.3.1.1. Method for the Synthesis of the Cyclic Peptide Analogue

The synthesis of the irreversible cyclic peptide analogues is performed following the same procedure and by replacing the bis-imidoester linker with the corresponding bis-succinimide ester crosslinker, for example disuccinimidyl adipate, pimelidate, and suberate respectively. These linkers react with amino groups to form alkaline stable amide bonds (FIG. 26).

### 5.4. CONSIDERATIONS ON THE PROPERTIES OF REVERSIBLE CYCLIC PEPTIDES AND LIBRARIES THEREOF

Sections 5.2 and 5.3 of this disclosure have presented general methods for the synthesis of cyclic peptides whose structure can be returned to linear by applying specific conditions. Without meaning to narrow the broadness/scope of our method, the reversible cyclic peptides presented in this disclosure are cyclic depsipeptides and cyclic amidine-peptides, in which the cleavable tether is an ester bond and an amidine bond respectively. Both these bonds are cleaved in alkaline conditions. These conditions, as well as any condition chosen to linearize analogous reversible cyclic peptides, are to be orthogonal to both the conditions employed for peptide synthesis and for library screening. This is to ensure that the peptides maintain a cyclic structure after synthesis and through the whole process of library screening and only after the selection of leads and only when desired, the cleavable tether is actually broken and the peptide returned to its linear structure. Some of the proposed chemistries afford linearized peptides with a primary amino terminus, while some others return linearized peptides with an —OH group in place of the N-terminus. While the former can be sequenced by means of both Edman degradation and single step MS/MS, the latter can be analyzed only by MS-based techniques or the like.

All the proposed chemistries also allow to cleave the peptide from the solid support under the same conditions employed for linearization. Without limiting the broadness of the invention, this disclosure proposes to couple the peptide to the resin via ester bond, but other analogous solutions can be arranged. As released in solution the peptide can be sequenced by a variety of solution phase techniques, like ESI-MS/MS. The proposed methods, however, allow as well to maintain the linearized peptide on solid phase, just by avoiding the use of HMBA linker, or the like, and coupling the “key-stone” tri-functional amino acid onto an amino group by peptide bond. In this case, the peptide can be sequence by Edman degradation, when allowed, or by other solid phase techniques, like MALDI-TOF/TOF.

Furthermore, the step of peptide synthesis performed by conventional Fmoc/tBu strategy, as is mentioned in each method in sections 5.2 and 5.3, allows the production of one sequence as well as of a combinatorial one-bead-one-peptide (OBOP) library by split-and-pool method. Lam, K. S., Lebl, M., and Krchnak, V. (1997) The “one-bead-one-compound” combinatorial library method. Chem. Rev. 97, 411-448. All the proposed chemistries also enable a wide range of spatial diversity, in addition to the chemical diversity imparted by the primary sequence of the peptides in question. The spatial diversity can be obtained by judiciously varying the position of “key stone” tri-functional amino acid as well as by using different sequence lengths. Different cyclization geometries resulting in loops of different sizes have been described in literature. Perlman Z. E., Bock J. E., Peterson J. R. and Lokey R. S. (2005), Geometric diversity through permutation of backbone configuration in cyclic peptide libraries, Bioorg. Med. Chem. Lett. 15 5329-5334. FIG. 27 illustrates how the location of the “key stone” tri-functional amino acid (glutamic acid in the figure) along a linear peptide chain can result in widely different constraints within the peptide after the cyclization.

Furthermore, while it is advantageous that cyclization adds spatial diversity to the peptide library, it must also be considered that the resulting peptide library can be very large, thereby significantly increasing the time to screen an entire library to identify binders for a given target protein. To reduce the size of the library, it is possible, within the presented methods, to create constrained libraries comprising only those amino acids that are known to play a predominant role in the interaction with the target protein. In fact it has been shown that, despite the relative large size of a protein-protein binding interface, single amino acids can contribute a large fraction of the total change in free energy of binding to the interface (90). Clackson, T. & Wells, J. A. (1995). A hot spot of binding energy in a hormone-receptor interface. Science 267, 383-386. These regions are referred to as “hotspots”.

### 5.5. ADDITIONAL MONOMERS AND LIBRARIES OF COMPOUNDS

Preparation and screening of combinatorial chemical libraries are well known to those of skill in the art. Such combinatorial chemical libraries include, but are not limited to, peptide libraries (see, e.g., U.S. Pat. No. 5,010,175 (Rutter and Santi), Furka, 1991, Int. J. Pept. Prot. Res., 37:487-493; and Houghton et al., 1991, Nature, 354:84-88). Other chemistries for generating chemical diversity libraries can also be used. Such chemistries include, but are not limited to: U.S. Pat. Nos. 6,075,121 (Bartlett et al.) peptoids; 6,060,596 (Lerner et al.) encoded peptides; 5,858,670 (Lam et al.) random bio-oligomers; 5,288,514 (Ellman) benzodiazepines; 5,539,083 (Cook et al.) peptide nucleic acid libraries; 5,593,853 (Chen and Radmer) carbohydrate libraries; 5,569,588 (Ashby and Rine) isoprenoids; 5,549,974 (Holmes) thiazolidinones and metathiazanones; 5,525,735 (Takarada et al.) and 5,519,134 (Acevado and Hebert) pyrrolidines; 5,506,337 (Summerton and Weller) morpholino compounds; 5,288,514 (Ellman) benzodiazepines; diversomers such as hydantoins, benzodiazepines and dipeptides (Hobbs et al., 1993, Proc. Nat. Acad. Sci. USA, 90, 6909-6913), vinylogous polypeptides (Hagihara et al., 1992, J. Amer. Chem. Soc., 114, 6568), nonpeptidal peptidomimetics with glucose scaffolding (Hirschmann et al., 1992, J. Amer. Chem. Soc., 114, 9217-9218), analogous organic syntheses of small compound libraries (Chen et al., 1994, J. Amer. Chem. Soc., 116:2661 (1994)), oligocarbamates (Cho et al., 1993, Science, 261, 1303 (1993)), and/or peptidyl phosphonates (Campbell et al., 1994, J. Org. Chem., 59:658), nucleic acid libraries (see Ausubel, Berger and Sambrook, all supra); antibody libraries (see, e.g., Vaughn et al., 1996, Nat. Biotech., 14(3):309-314, carbohydrate libraries, e.g., Liang et al., 1996, Science, 274:1520-1522, small organic molecule libraries (see, e.g., benzodiazepines, Baum, 1993, C&EN, Jan. 18, page 33. Devices for the preparation of combinatorial libraries are commercially available (see, e.g., 357 MPS, 390 MPS, Advanced Chem Tech, Louisville Ky., Symphony, Rainin, Woburn, Mass., 433 A Applied Biosystems, Foster City, Calif., 9050 Plus, Millipore, Bedford, Mass.). In addition, numerous combinatorial libraries are themselves commercially available (see, e.g., ComGenex (Princeton, N.J.), Asinex (Moscow, RU), Tripos, Inc. (St. Louis, Mo.), ChemStar, Ltd., (Moscow, RU), 3D Pharmaceuticals (Exton, Pa.), Martek Biosciences (Columbia, Md.), etc.).

Specific examples of compound libraries include: cyclic peptides (Nauman et al., 2008, ChemBioChem 9, 194-197); HDAC inhibitors from a cyclic α3β-tetrapeptide library (Olsen and Ghadiri, 2009, J. Med. Chem. 52(23), 7836-7846), the contents of all of which are hereby incorporated by reference in their entireties.

### 5.6. LIBRARY SCREENING

A solid phase library of reversible cyclic peptides synthesized as explained in sections 5.2 and 5.3 can be screened for on-bead ligands that bind to a target biological. Without limiting the scope of the invention, this disclosure suggests the use of a radiological screening comprising: (i) incubation of the library with the target protein that has been radiolabeled; (ii) loading of library beads on the agarose gel; (iii) incubation of the beads on agarose with an autoradiographic film; (iv) development of the radiographic film; (v) identification of positive leads by overlapping the agarose gel and the radiographic film; and (vi) excision and screening of positive leads. The selected beads are then subjected to the alkaline treatment reported in section 5.7. See Mondorf and Carbonell 1998.

### 5.7. LINEARIZATION AND CLEAVAGE OF REVERSIBLE CYCLIC PEPTIDES AND MS/MS ANALYSIS OF THE LINEARIZED PEPTIDES FOR SEQUENCE DETERMINATION

Based on the protocols reported at 6.1 and 6.3, it is possible, with a single alkaline treatment, to attain at once both the opening of the peptide ring and the release of the linearized sequence in solution from an aliquot of beads or from a single bead selected through library screening against a target biological, as explained at 6.5. The cleavage is performed with an aqueous-organic alkaline to ensure the extraction of the whole amount of peptide from each single bead. The procedures indicated herein have the sole purpose of exemplifying the procedure of peptide cleavage and linearization with good indications, but they do not imply any restriction to the broadness of the method.

After rinsing, an aliquot of resin is treated with an alkaline solution of acetonitrile in water. An appropriate volume of pure TFA was added to the cleavage solution to neutralize the pH. The cleaved samples are analyzed by RP-HPLC to estimate the purity of the cleaved linearized peptide. The cleaved samples can also be analyzed by ESI-MS/MS.

Also, to simulate the process of sequence identification from a single bead selected from library screening, a single bead of the resin is treated with a small volume of an alkaline solution of acetonitrile in water. The treatment is preferably performed at low temperature to avoid the evaporation of a significant amount of the cleaving mixture. The resulting sample is neutralized with formic acid, concentrated and desalted. After dilution, if needed, the sample is analyzed by ESI-MSMS.

In alternative, in case when the reversible cyclic peptide can only be linearized but not cleaved, to simulate the process of sequence identification from a single bead selected from library screening, a solid-phase technique of peptide sequencing can be used. A single bead is incubated with a small volume of an alkaline solution of acetonitrile in water. After rinsing, the bead is mixed with MALDI resin and analyzed by MALDI-TOF/TOF.

Without limiting the broadness of the invention, this disclosure further proposes the adoption of a bioinformatic approach to perform an unbiased determination of the peptide sequences from the selected beads. The method employs software to compare the spectral data obtained by MS/MS analysis with a library of theoretical spectra generated according to model fragmentation patterns. The selection of the sequences from the list of possible matches provided by the software is to be based on considerations of peptide composition and consensus homology.

Without limiting the scope of the invention, this disclosure foresees that in a software-based automated work of spectral analysis and sequence identification, the tallest peak among those with highest molecular weight is automatically selected and sent for subsequent fragmentation to determine the amino acid sequence. As shown in the result section herein, the heaviest compound corresponding to the linearized peptide is the one present in the highest amount. Therefore the highest peak, which is always the one corresponding to the highest molecular weight, and therefore the one corresponding to the linearized peptide, is to be selected for further fragmentation in order to determine the amino acid composition.

In the following section, the symmetrical sequence VVWVV, the neutravidin-binding sequence DRASPY, and the antibody-binding sequence WFRHY (SEQ ID NO:16-18) are presented as examples.

Unless defined otherwise, all technical and scientific terms used herein have the same meaning as commonly understood by one of ordinary skill in the art to which this invention belongs. The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention. In particular, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### 6. EXAMPLES

### 6.1. Synthesis of Cyclic Depsipeptides on HMBA-Chemmatrix Resin

Materials and Methods

Protected amino acids and coupling agents for peptide synthesis were purchased from ChemPep Inc. (Wellington, Fla., USA). Diisopropylethylamine (DIPEA), trifluoroacetic acid (TFA), triisopropylsylane (TIPS), ethanedithiol (EDT), aqueous 1N NaOH, PBS pH 7.4 buffer were from Sigma Aldrich (Saint Louis, Mo., USA). Anhydrous solvents, N,N-dimethylformamide (DMF), dichloromethane (DCM), HPLC grade acetonitrile and water, sodium chloride, acetic acid glacial, 85% v/v phosphoric acid, dimethyl adipimidate and succinimidyl glutarate (DSG) were from Fisher Scientific (Pittsburgh, Pa., USA). PEG—based HMBA-ChemMatrix resin (functional density of 0.6 meq/g) was purchased from PCAS Biomatrix Inc. (Saint-Jean-sur-Richelieu, Quebec, Canada). Toyopearl AF-Amino-650M resins were purchased from Tosoh Bioscience (King of Prussia, Pa., USA). Human polyclonal IgG was from Equitech-Bio (Kerrville, Tex., USA).

In this example six model peptides, namely cyclo[Lac-VVWVV-E], cyclo[Lac-A-VVWVV-E], cyclo[Lac-DRASPY-E], cyclo[Lac-A-DRASPY-E], cyclo[Lac-A-VWV-E-VV], and cyclo[Lac-A-WFRHY-E] (SEQ ID NO:19-24) were synthesized according to the method proposed in section 5.2.3.1. The peptides were synthesized on 75-150 micron diameter HMBA-ChemMatrix® resin (substitution level of 0.6 mmol/g). Each coupling step was conducted for 25 min in a polypropylene tube fitted with a Teflon frit under continuous nitrogen flow. To enhance the reaction rate, sonication was carried out using a Branson ultrasonic bath (Model 1510; sonicating frequency 40 kHz) and the temperature was maintained at 35° C. The synthesis of each sequence was started with 250 mg of resin and consisted of the following steps: (i) two couplings were performed with 3 eq. (molar excess as compared to density of HMBA linker) of Fmoc-Glu(OAll)-OH, 3 eq. of 2-(7-Aza-1H-benzotriazole-1-yl)-1,1,3,3-tetramethyluronium hexafluorophosphate (HATU) and 6 eq. of diisopropylethylamine (DIPEA) in 2.5 mL of dry DMF. (ii) In order to cap the unreacted HMBA linker, an acetylation step with 50 eq. acetic anhydride and DIPEA solution in DMF was carried out for 30 min at room temperature. (iii) The Fmoc protection was then removed by incubating the resins with 5 mL of 20% piperidine in DMF solution for 30 min. The four linear peptide sequences, namely VVWVV, DRASPY, AVVWVV, ADRASPY, AVWVEVVV, and AWFRHY (SEQ ID NO:15-16, 25-28) were synthesized via conventional Fmoc/tBu strategy. An anhydrous DMF solution (2.5 mL) of 3 eq. Fmoc-amino acid, 3 eq. 2-(6-Chloro-1H-benzotriazole-1-yl)-1,1,3,3-tetramethylaminium hexafluorophosphate (HCTU) and 6 eq. DIPEA was added to the resin. Albericio F., Chinchilla R., Dodsworth D. J. and Najera C. (2001), New trends in peptide coupling reagents, ChemInform 32, 31, 203-313; Han S.-Y. and Kim Y.-A. (2004), Recent development of peptide coupling reagents in organic synthesis, Tetrahed. 60, 2447-2467. Three couplings were performed for each amino acid to saturate all the available amino groups, as monitored by the Kaiser test. Kaiser E., Colescott R. L., (1970) Color test for detection of free terminal amino groups in the solid-phase synthesis of peptides. Anal. Biochem., 34, 595-598. The Fmoc protection was maintained on the last amino acid. (v) The allyl ester protection on the carboxyl group of the glutamic acid was removed within minutes by treatment with 0.1 eq. of tetrakis(triphenylphosphine)palladium(0) and 10 eq. phenylsilane as a scavenger in DCM. The resin was then rinsed three times for 15 min with 0.02 eq. of sodium diethyldithiocarbamate in DMF to remove the palladium catalyst. Llobet A. I., lvarez M. and Albericio F., (2009), Amino Acid-Protecting Groups, Chem. Rev. 109, 2455-2504. (vi) Allyl lactate was selected as cleavable linker and coupled by ester bond formation on the carboxyl group of glutamic acid by HATU chemistry. Albericio F., Chinchilla R., Dodsworth D. J. and Najera C. (2001), New trends in peptide coupling reagents, ChemInform 32, 31, 203-313. Han S.-Y. and Kim Y.-A. (2004), Recent development of peptide coupling reagents in organic synthesis, Tetrahed. 60, 2447-2467. Three couplings were performed with 3 eq. of allyl lactate, 3 eq. of HATU and 6 eq. of DIPEA in 1 mL of dry DMF. (vii) The allyl ester protection on the carboxyl group of the lactic acid was removed with the aforementioned Pd-based treatment. (viii) The Fmoc group on the final amino acid was then removed with 5 mL of 20% piperidine in DMF solution for 30 min. The Kaiser test confirmed the presence of free amino groups. (ix) The peptide cyclization was performed by coupling the carboxyl group of the linker (lactic acid) to the peptide N-terminus using a solution of 3 eq. HATU and 6 eq. DIPEA in dry DMF for 45 minutes. A second coupling was repeated to ensure completion of reaction. A Kaiser test indicated the absence of free amino groups therefore confirming the formation of the peptide ring. (x) Finally, peptide deprotection was performed using a cleavage cocktail containing TFA/TIPS/H2O/EDT (94/3/2/1) for 1.5 hours.

### 6.2. Synthesis of Cyclic Peptides on Toyopearl Amino Resin

In this example two sequences, namely cyclo[Lac-A-DRASPY-E] (SEQ ID NO:21), and cyclo[A-A-DRASPY-E] (SEQ ID NO:29) were synthesized on 100-150 micron diameter Toyopearl AF-Amino-650M resin (substitution level of 0.4 mmol/g) under the same conditions as above. The synthesis was started on a single batch of 300 mg of resin and comprised the following steps: two couplings were performed with 3 eq. (molar excess as compared to density of amino groups) of Fmoc-Glu(OAll)-OH, 3 eq. HCTU, and 6 eq. of diisopropylethylamine (DIPEA) in 2.5 mL of anhydrous DMF. The Fmoc protection was then removed by incubating the resins with 5 mL of 20% piperidine in DMF solution for 30 min. The linear sequence ADRASPY (SEQ ID NO:26) was synthesized via conventional Fmoc/tBu strategy. The batch of resin was split in two aliquots, a) and b). Aliquot a) was subjected to the above mentioned procedure of coupling the allyl lactate, cleavage of allyl and Fmoc protections, cyclization of the depsipeptide and final cleavage of side protecting groups. Aliquot b) was treated with 20% piperidine in DMF to remove the Fmoc protection and then subjected to an additional coupling of Fmoc-Ala-OH. Then the allyl ester protection on the carboxyl group of glutamic acid and the Fmoc protection on Ala were cleaved sequentially using the Pd-based treatment and 20% piperidine as described above. Final peptide cyclization and deprotection were carried out as reported above.

### 6.3. Synthesis of Cyclic Amidine-Peptides on HMBA-ChemMatrix Resin

In this example the cyclic amidine-peptides cyclo[AVVWVVK-Adipimidate], cyclo[ADRASPYK-Adipimidate] and cyclo[AMWFPHYK-Adipimidate] (SEQ ID NO:30-32) were synthesized according to the method proposed in section 5.3.1. The peptides were synthesized on HMBA-ChemMatrix® resin (substitution level of 0.6 mmol/g) and Toyopearl AF-Amino-650M resin (substitution level of 0.4 mmol/g). Each coupling step was conducted for 25 min in a polypropylene tube fitted with a Teflon frit under continuous nitrogen flow. To enhance the reaction rate, sonication was carried out using a Branson ultrasonic bath (Model 1510; sonicating frequency 40 kHz) and the temperature was maintained at 35° C. The synthesis was started with 250 mg of both resin and consisted of the following steps: (i) two couplings were performed with 3 eq. (molar excess as compared to density of HMBA linker) of Nα-Fmoc-NE-allyloxycarbonyl lysine Fmoc-Lys(Aloc)-OH, 3 eq. of 2-(7-Aza-1H-benzotriazole-1-yl)-1,1,3,3-tetramethyluronium hexafluorophosphate (HATU) and 6 eq. of diisopropylethylamine (DIPEA) in 2.5 mL of dry DMF. (ii) In order to cap the unreacted HMBA linker, an acetylation step with 50 eq. acetic anhydride and DIPEA solution in DMF was carried out for 30 min at room temperature. The Fmoc protection was then removed by incubating the resins with 5 mL of 20% piperidine in DMF solution for 30 min. The linear peptide sequences, namely AVVWVV, ADRASPY (SEQ ID NO:25-26) and AMWFPHY (SEQ ID NO:33), were synthesised via conventional Fmoc/tBu chemistry. An anhydrous DMF solution (2.5 mL) of 3 eq. Fmoc-amino acid, 3 eq. HCTU and 6 eq. DIPEA was added to the resin. Two coupling steps were performed for each amino acid to saturate all the available amino groups, as monitored by the Kaiser test. The Fmoc protection was maintained on the last amino acid. The resin was washed with DMF (2×5 min) and DCM (2×5 min). The allyloxycarbonyl protection on the ε-amino group of Lys was removed within minutes with 0.1 eq. of tetrakis(triphenylphosphine)palladium(0) and 10 eq. phenylsilane as scavenger in DCM. The resin was then rinsed with 0.02 eq. of sodium diethyldithiocarbamate in DMF (3×15 min). The Fmoc group on alanine was then removed with 5 mL of 20% piperidine in DMF solution for 30 min at room temperature. The resin was then rinsed with α-amino specific crosslinking buffer (0.1M carbonate buffer, 0.2M triethanolamine, pH 8.0). After resin equilibration, 10 eq. of crosslinker dimethyl adipimidate (DMA) were added to the resin suspension and stirred for 30 min at room temperature. The resin was then rinsed with ε-amino specific crosslinking buffer (0.1M carbonate buffer, 0.2M triethanolamine, pH 10.0). After resin equilibration, 10 eq. of crosslinker DMA were added to the resin suspension and stirred for 30 min. Finally, peptide deprotection was performed using a cleavage cocktail containing TFA/TIPS/H2O/EDT (94/3/2/1 v/v/v/v) for 1.5 hours.

### 6.4. Synthesis of Cyclic Peptides on Toyopearl Amino Resin

In this example the sequences cyclo[ADRASPYK-Adipate] and cyclo[AMWFPHYK-Adipate] (SEQ ID NO:31-32) were synthesized on 100-150 micron diameter Toyopearl AF-Amino-650M resin (substitution level of 0.4 mmol/g) under the same conditions as above. The synthesis was started on a single batch of 300 mg of resin and comprised the following steps: two couplings were performed with 3 eq. (molar excess as compared to density of amino groups) of Fmoc-Lys(pNZ)—OH, 3 eq. HCTU, and 6 eq. of diisopropylethylamine (DIPEA) in 2.5 mL of anhydrous DMF. The Fmoc protection was then removed by incubating the resins with 5 mL of 20% piperidine in DMF solution for 30 min. The linear sequences ADRASPY and AMWFPHY (SEQ ID NO:26,33) were synthesized by conventional Fmoc/tBu strategy. The Fmoc group on alanine was then removed with 5 mL of 20% piperidine in DMF solution for 30 min at room temperature. The linker p-Nitrobenzyl adipate was coupled to the peptide N-terminus: two couplings were performed with 3 eq. (molar excess as compared to density of amino groups) of p-Nitrobenzyl adipate, 3 eq. HCTU, and 6 eq. of DIPEA in 2.5 mL of anhydrous DMF. The p-Nitrobenzyloxycarbonyl protecting group and the p-Nitrobenzyl ester protecting group were removed respectively from the ε-amino group of Lysine and the adipic acid linker with tin (II) chloride in slightly acidic DMF (1.6 mM HCl in dioxane)). The peptide cyclization was performed by coupling the carboxyl group of the adipic acid linker to the ε-amino group of Lysine with a solution of 3 eq. HATU and 6 eq. DIPEA in dry DMF for 45 minutes. A second coupling was repeated to ensure completion of reaction. A Kaiser test indicated the absence of free amino groups therefore confirming the formation of the peptide ring. Finally, peptide deprotection was performed using a cleavage cocktail containing TFA/TIPS/H2O/EDT (94/3/2/1) for 1.5 hours.

6.4.1. Screening Simulation of a Library of Reversible Cyclic Peptides Using the Mondorf-Carbonell Radiological Procedure

The simulation of library screening according to the Mondorf-Carbonell method described at section 5.5 was performed on the resins cyclo[Lac-A-DRASPY-E]-HMBA-ChemMatrix and cyclo[ADRASPYK-Adipimidate]-HMBA-ChemMatrix (SEQ ID NO:21, 31) to demonstrate that the method has true potential towards the successful library screening and the effective identification of positive leads. Neutravidin was radiolabeled according to the procedure reported by Mondorf et al. using ¹⁴C formaldehyde by reductive amination. The prospected extent of radiolabeling was 5%. Mondorf Carbonell 1998.

Two aliquots of cyclo[Lac-A-DRASPY-E]-HMBA-ChemMatrix resin and cyclo[Lac-A-VVWVV-E]-HMBA-ChemMatrix (SEQ ID NO:20-21) resin and were mixed in a 50:50 proportion. 10 mg of the resin mix was swollen in 20% methanol in water for 30 min and then rinsed with PBS, pH 7.4 (3×5 min). The resin slur was then mixed with 200 μl of 5 mg/mL ¹⁴C-labelled Neutravidin. After 2 hrs of incubation the supernatant was decanted and the resin was washed with PBS buffer containing 0.2M NaCl and 0.1% (w/v) Tween 20 until the radioactivity reached the baseline level. The washed resin was then mixed with 20 mL of 1% low melting agarose solution. The mixture was swirled to form a uniform suspension and then the slurry was poured on 16×18 cm gelbond film to form a monolayer of beads. This gel was air-dried overnight in a hood. The dried agarose gel was then exposed to photographic film for 5 days at room temperature. The photographic film was then developed, the dark spot located on the film and the corresponding beads selected.

The same procedure was repeated with the resins cyclo[ADRASPYK-Adipimidate]-HMBA-ChemMatrix and cyclo[AVVWVVK-Adipimidate]-HMBA-ChemMatrix (SEQ ID NO:30-31) using ¹⁴C-radiolabeled human polyclonal IgG as target biological.

6.4.2. Linearization and Cleavage of Reversible Cyclic Peptides and MS/MS Analysis of the Linearized Peptides for Sequence Determination

A 5 mg aliquot of the resins synthesized at 6.1 and 6.3 were rinsed with 20:80 acetonitrile:water and then treated with 1 mL of 0.1M NaOH in 20:80 acetonitrile:water for 20 min at room temperature. An appropriate volume of pure TFA was added to the cleavage solution to neutralize the pH. RP-HPLC Analysis of the cleavage samples was performed by with a C18 column, running a linear elution gradient from 95/5 to 80/20 water+0.1% TFA/acetonitrile+0.1% TFA, over 30 min. Absorbance was monitored by UV absorption at 280 nm. Each sample was also analyzed by ESI-MS/MS.

Also, to simulate the process of sequence identification from a single bead selected from library screening, a single bead of the resins synthesized at 6.1 and 6.3 was rinsed with 20:80 acetonitrile:water and then treated with 0.1 mL of 0.1M NaOH in 20:80 acetonitrile:water for 20 min at 4 C. The resulting sample was concentrated and desalted using a C18 ZipTip pipette tip. The sample was diluted with 50 μL of 0.1% formic acid in water to be analyzed with ESI-MSMS.

6.4.3. Comparison of the Binding Properties of Reversible Vs. Irreversible Cyclic Peptides Synthesized on Chromatographic Resins

As the peptide sequences are selected and identified in their reversible cyclic form but they are employed in their corresponding irreversible cyclic form, it is necessary to confirm that the two forms present the same binding behavior towards the protein target. To this end, the following chromatographic screening was performed.

Thirty-five milligrams of cyclo[Lac-A-DRASPY-E]-Toyopearl resin and cyclo[A-A-DRASPY-E]-Toyopearl (SEQ ID NO:21, 29) resin were packed in a 30 mm×2.1 mm I.D. Microbore column (0.1 mL) and swollen with 20% v/v methanol. One hundred microliters of feed sample, namely Neutravidin 5 mg/mL in PBS pH 7.4, was loaded onto the column at a flow rate of 0.05 mL/min (87 cm/h). The column was washed with 2 mL of equilibration buffer at a flow rate of 0.2 mL/min (348 cm/h). Elution was then performed with 4 mL of 0.2M glycine buffer pH 2.5 at the flow rate of 0.4 mL/min (696 cm/h). Cleaning and regeneration were performed by 4 mL of 0.85% phosphoric acid. The effluent was monitored by absorbance at 280 nm. Fractions were collected and concentrated five times by centrifugation at 4° C., 20817×g for 30 min using an Amicon® Ultra centrifugal filter (3000 MWCO, Ultracel®, Millipore, Billerica, Mass., USA).

Thirty five milligrams of cyclo[AMWFRHYK-Adipimidate]-Toyopearl *** and cyclo[AMWFPHYK-Adipate]-Toyopearl (SEQ ID NO:32, 34) resin was packed in a 30 mm×2.1 mm I D Microbore column (0.1 mL) and swollen with 20% v/v methanol. One hundred microliters of feed sample, namely human polyclonal IgG 5 mg/mL in PBS pH 7.4, was loaded onto the column at a flow rate of 0.05 mL/min (87 cm/h). The column was washed with 2 mL of equilibration buffer at a flow rate of 0.2 mL/min (348 cm/h). Elution was then performed with 4 mL of 0.2M acetate buffer pH4 at the flow rate of 0.4 mL/min (696 cm/h). Cleaning and regeneration was performed by 4 mL of 0.85% phosphoric acid. The effluent was monitored by absorbance at 280 nm. Fractions were collected and concentrated five times by centrifugation at 4° C., 20817×g for 30 min using an Amicon® Ultra centrifugal filter (3000 MWCO, Ultracel®, Millipore, Billerica, Mass., USA).

### 6.5. Results and Discussion

6.5.1. Screening Simulation of a Library of Reversible Cyclic Peptides Using the Mondorf-Carbonell Radiological Procedure

Two reversible cyclic peptide resins were chosen to simulate the process of library screening against a biological for the selection of candidate beads. The two cyclic depsipeptide resins cyclo[Lac-A-DRASPY-E]-HMBA-ChemMatrix and cyclo[Lac-A-VVWVV-E]-HMBA-ChemMatrix (SEQ ID NO:20-21) were employed to simulate library screening with Neutravidin, while the two cyclic amidine-peptide resins cyclo[AMWFRHYK-Adipimidate]-HMBA-ChemMatrix and cyclo[AVVWVVK-Adipimidate]-HMBA-ChemMatrix (SEQ ID NO:30,34) were employed to simulate library screening with human polyclonal IgG. The sequences DRASPY and WFRHY are known ligands for the target proteins, while the sequence VVWVV (SEQ ID NO:16-18) is selected as a negative control.

The screening procedure followed the method set forth in sections 5.5 and 6.5. In both cases, an aliquot of 10 mg of 50:50 mix of the ligand resin and the negative control resin were equilibrated with PBS and mixed with 5% ¹⁴C-radiolabeled target, namely Neutravidin and IgG. After incubation, the resin was washed with PBS buffer containing 0.2M NaCl and 0.1% (w/v) Tween 20. A volume of 250 mL of washing solution was necessary in order for the radioactivity to reach the baseline level. Beads were then suspended in agarose and plated on an agarose film. After drying the gel, beads were contacted with a photographic film for five days. The developed films indicated that the ligand resins cyclo[Lac-A-DRASPY-E]-HMBA-ChemMatrix and cyclo[Lac-A-VVWVV-E]-HMBA-ChemMatrix (SEQ ID NO:20-21) bound a sufficient amount of radiolabeled target protein to impress a dark spot on the developed radiographic film, while the negative control resins carried an amount of radioactive protein below the detection limit. In both cases, the difference between the levels of radioactivity carried by the resin was enough to discriminate the ligand resin beads from the negative control beads.

This indicates that the proposed method can be successfully applied for identification of target protein ligands. This solid-phase screening technique seems to be much better than the liquid phase screening of biological libraries that have been often proposed for the identification of cyclic peptide ligands. In fact, one-bead-one-peptide libraries are a very efficient choice for identifying affinity peptide ligands (74). Lam, K. S., Lebl, M., and Krchnak, V. (1997) The “one-bead-one-compound” combinatorial library method. Chem. Rev. 97, 411-448. The peptide ligands found to bind to the target protein from these one-bead-one-peptide libraries can be directly used in the bead assay to detect the target protein, as shown in the sections 6.8.4 and 6.8.5.

6.6. RP-HPLC and ESI-MS/MS Analysis of Cyclic Depsipeptides

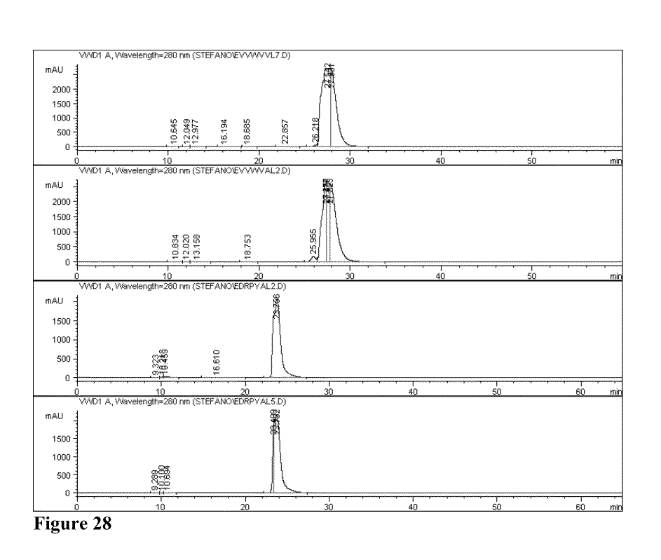

The cyclic depsipeptides sequences cyclo[Lac-VVWVV-E], cyclo[Lac-A-VVWVV-E], cyclo[Lac-A-DRASPY-E], and cyclo[Lac-A-VWV-E-VV] (SEQ ID NO:19-22) synthesized on HMBA-ChemMatrix resin were cleaved and linearized in alkaline conditions (0.1M NaOH in 20:80 acetonitrile:water) and analyzed by RP-HPLC (C18) to estimate the purity of the depsipeptides on solid-phase. The RP-HPLC results (FIG. 28) indicate that the cyclic depsipeptides synthesized on solid phase are highly pure and form the most abundant species on each resin. This is critically important towards the success of the library screening. The high purity of the library, in fact, lowers the risk of identifying false positive and significantly facilitates the process of sequence identification.

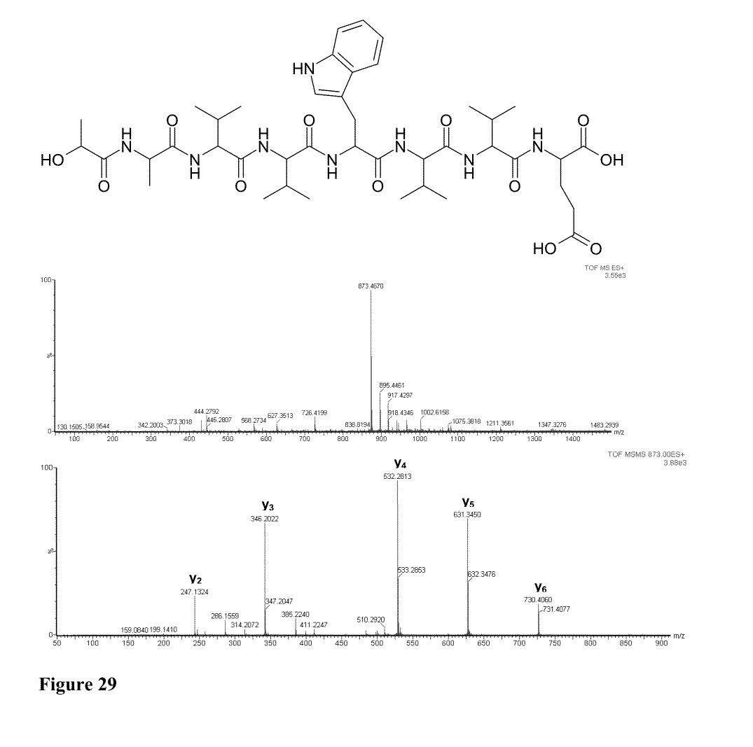

FIG. 29 shows the structure and the ESI-MS/MS analysis of linearized sequences Lac-A-VVWVV-E and Lac-VVWVV-E (SEQ ID NO:10-11) cleaved from a single bead. As the MS and MS/MS spectra clearly indicate, both peptides are highly pure and the generation of peptide fragments ions mainly of the y-series is very regular and enables very accurate sequence identification. It is also noticed that, because the peptide has an —OH group in place of a primary amine on the N-terminus, the first y fragment comprises Lac and its neighboring amino acid, respectively A and V. in fact, the heaviest ions appearing in the MS/MS spectra are respectively VVWVVE and VWVVE (SEQ ID NO:35-36). This indicates that having an additional amino acid between the Lac linker and the actual sequence lowers the computational effort of sequence identification, which is a significant advantage in the sequencing process of tens or hundreds of leads identified through library screening.

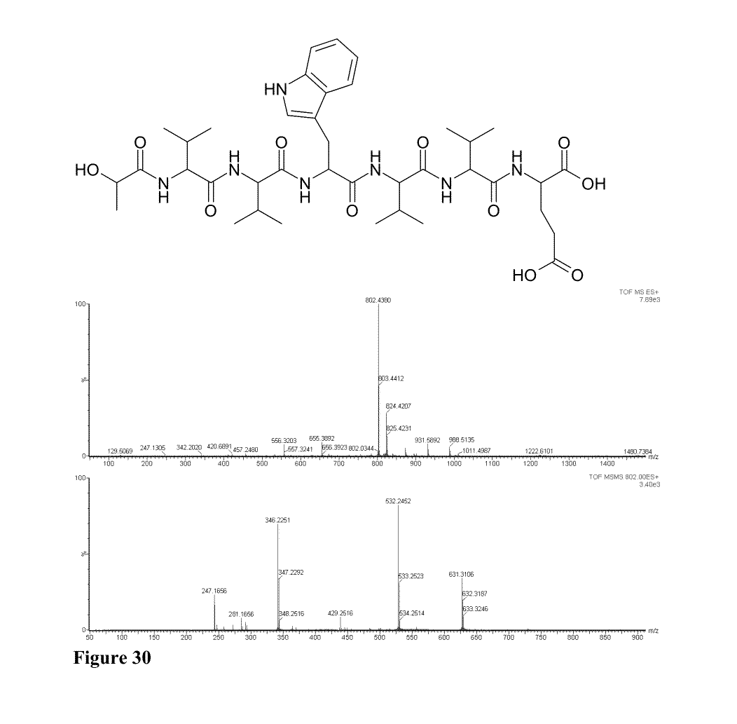

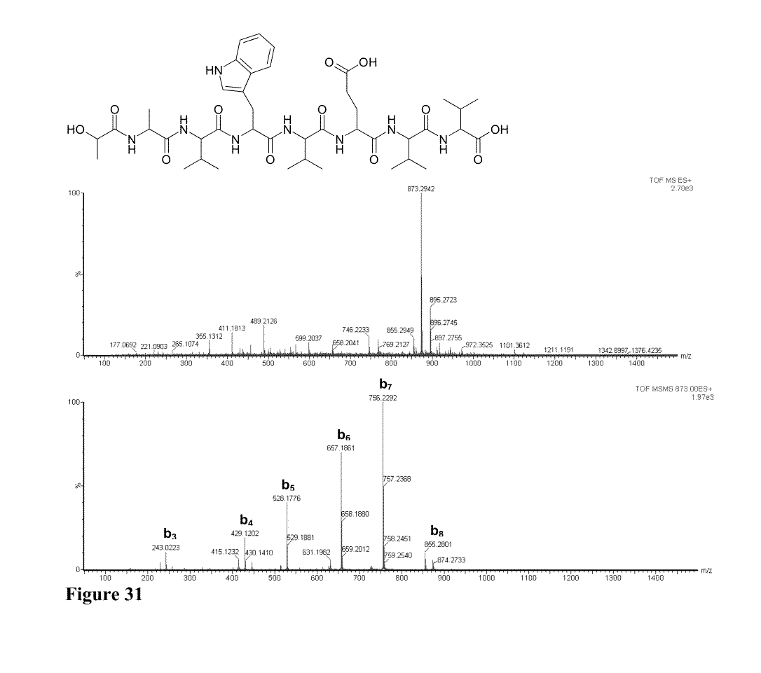

FIG. 30 shows the structure and the ESI-MS/MS analysis of linearized sequence Lac-A-VWV-E-VV (SEQ ID NO:12) from a single bead. This correspond to the structure portrayed in FIG. 31 demonstrates that spatial diversity in libraries of depsipeptides can be achieved by varying the position of the “keystone” tri-functional amino acid, in this case represented by glutamic acid. As the MS and MS/MS spectra clearly indicate, the peptides are highly pure and the generation of peptide fragments ions mainly of the y-series is very regular and enables very accurate sequence identification.

FIG. 32 shows the structure and the ESI-MS/MS analysis of linearized sequence Lac-A-DRASPY-E (SEQ ID NO:13) from a single bead. As the MS data indicate the peptide is highly pure. However, unlike the results reported in FIG. 32 the MS/MS analysis of the sequence affords a variety of fragmentation patterns that require computational tools for spectral analysis and sequence identification. For this purpose, a bioinformatic approach was adopted based on software that compares the experimental MS/MS spectra with a library of theoretical spectra generated according to model fragmentation patterns, and provides a list of matching sequence titles with scores of probability and matching quality. A database containing all the possible hexapeptide combinations framed between Lac-Ala- and -Glu, i.e. Lac-A-X₁X₂X₃X₄X₅X₆E, was created in FASTA format, as follows:

>XXXXXX sequence title
AAXXXXXXE sequence structure

The MS/MS spectrum of the sequence Lac-A-DRASPY-E (SEQ ID NO:13) was smoothed using a Savitzky-Golay filter and centered. The software analysis allowed the identification of the peptide sequence. FIG. 32 shows the MS/MS spectrum labeled by the software for peak identification.

### 6.7. RP-HPLC and ESI-MS/MS Analysis of Cyclic Amidine-Peptides

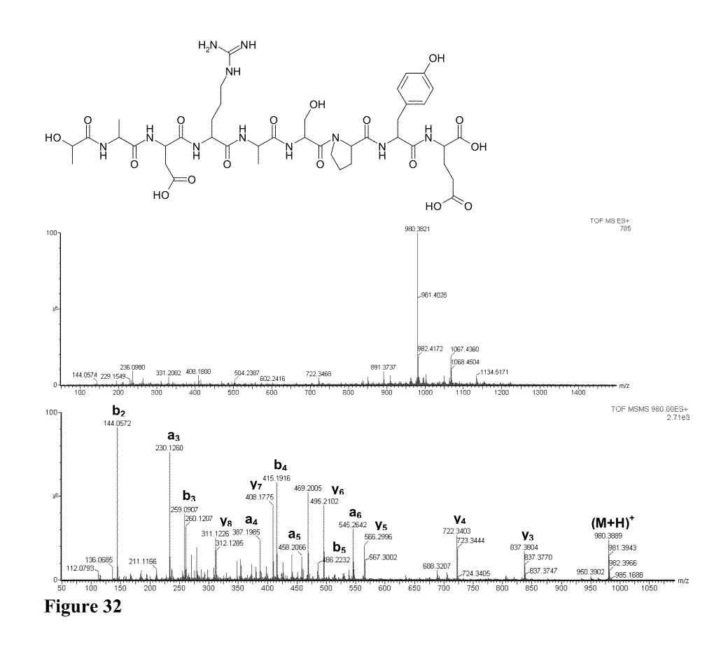

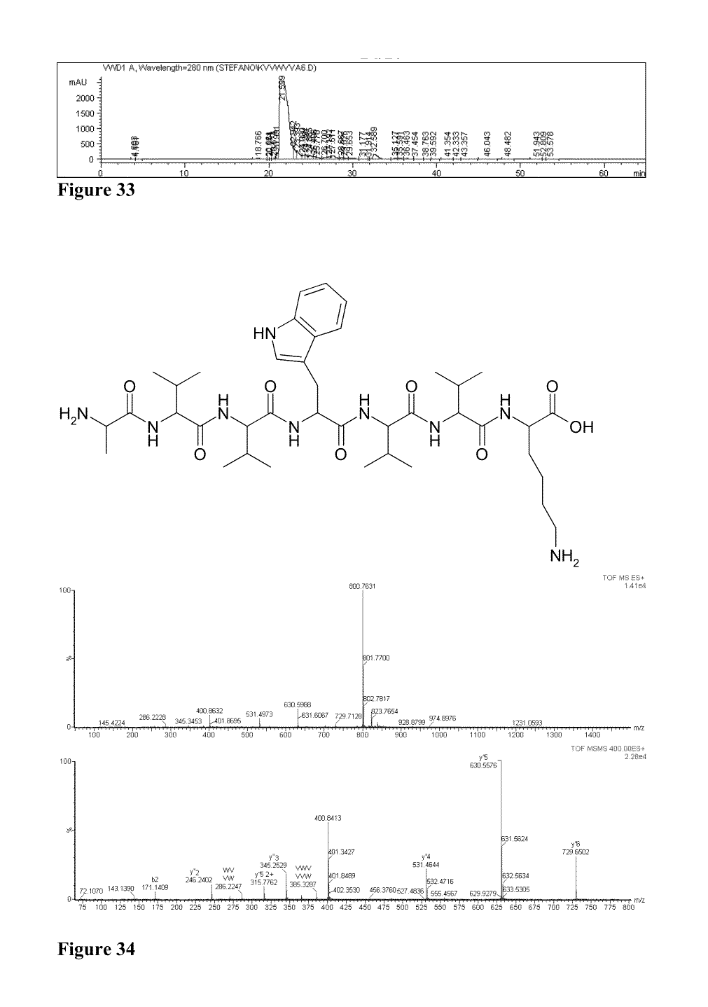

The cyclic amidine-peptide sequence cyclo[AVVWVVK-Adipimidate] (SEQ ID NO:30) synthesized on HMBA-ChemMatrix resin was cleaved and linearized in alkaline conditions (0.1M NaOH in 20:80 acetonitrile:water) and analyzed by RP-HPLC (C18) to estimate the purity of the depsipeptides on solid-phase. The RP-HPLC results (FIG. 33) indicate that the cyclic depsipeptides synthesized on solid phase are highly pure and form the most abundant species on each resin. This is critically important towards the success of the library screening. The high purity of the library, in fact, lowers the risk of identifying false positive and significantly facilitates the process of sequence identification.

FIG. 34 shows the structure and the ESI-MS/MS analysis of linearized sequences AVVWVVK (SEQ ID NO:15) cleaved from a single bead. As the MS and MS/MS spectra clearly indicate, the peptide is highly pure and the generation of peptide fragments ions mainly of the y-series is regular and enables accurate sequence identification.

### 6.8. Chromatographic Comparison of the Cyclic Depsipeptide and the Cyclic Peptide Versions of the Neutravidin-Binding Peptide Sequence DRASPY

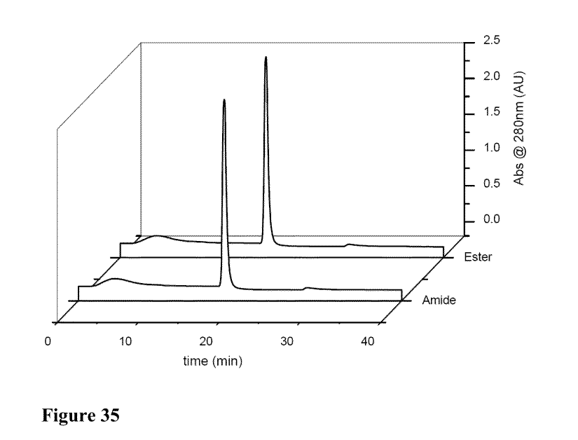

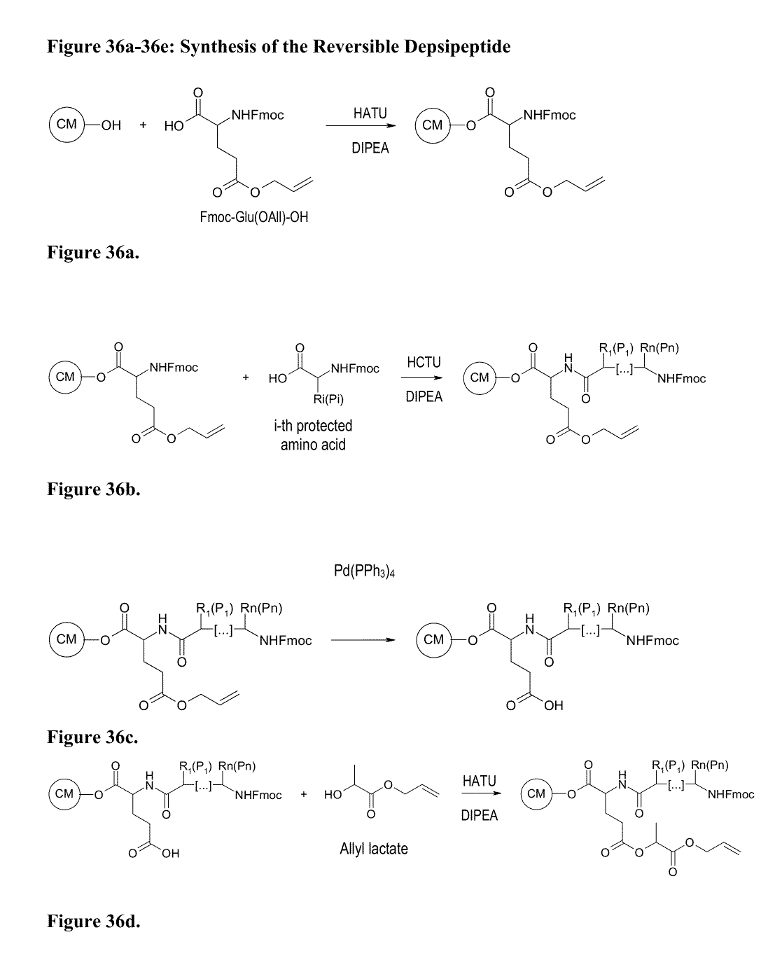

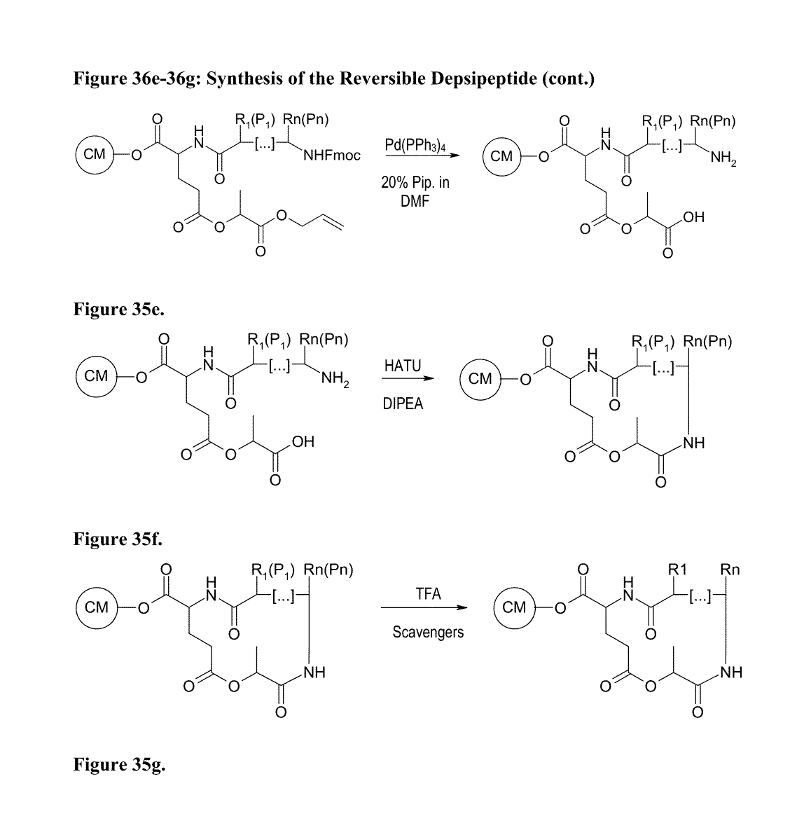

Cyclic depsipeptides are present in nature as very selective ligands and enzyme substrates. In many cases, however, the depside (ester) bond is found to play an active role in their biological activity and the homodetic cyclic peptide version of the same sequence can partially or completely loose the target affinity. In the technique proposed herein, the binding sequence is in a cyclic heterodetic form when is selected from a library of cyclic depsipeptides by screening with the target biological, and yet it is in a cyclic homodetic form when employed as a drug or as affinity ligand towards the same target biological. It is hence necessary to compare the binding property of the cyclic homodetic and heterodetic forms of the Neutravidin-binding sequence synthesised on a chromatographic resin. Two resins, cyclo[Lac-A-DRASPY-E]-Toyopearl resin and cyclo[A-A-DRASPY-E]-Toyopearl (SEQ ID NO:21, 29) resin, were tested on column for streptavidin binding under the same chromatographic conditions. PBS pH 7.4 and 0.2M Glycine pH 2.5 were chosen as binding and elution buffers respectively. The chromatogram reported in FIG. 35 clearly shows no difference between the cyclic depsipeptide and the cyclic peptide version of the same ligand sequence, indicating that the depside bond does not play any role in binding the target.

6.9. Chromatographic Comparison of the Cyclic Amidine-Peptide and the Cyclic Peptide Versions of the Antibody-Binding Peptide Sequence WFRHY

For the reasons discussed in section 6.8.3, as the cyclic structures formed by amidine and amide bond are slightly different, it is necessary to compare the binding behaviour of the two forms. The antibody-binding sequence WFRHY (SEQ ID NO:18) was synthesized in both amidine and amide versions on a chromatographic resin and tested on column for human polyclonal IgG binding under the same chromatographic conditions. PBS pH 7.4 and 0.2M Acetate pH 4.0 were chosen as binding and elution buffers respectively. The chromatographic results show (e.g., FIG. 35) no difference between the cyclic amidine-peptide and the cyclic peptide version of the same ligand sequence, indicating that the difference between the amidine bond and the amide bond does not play any role in binding the target.

The analogue cyclic peptide structures created by replacing the Lac-Ala tether with an Ala-Ala tether or by replacing the amidine bond with an amide bond are hence stable in alkaline conditions. This is a necessary requirement for cyclic peptides meant to be used as ligands for protein purification, as the regulations for affinity chromatography on industrial scale require the use of alkaline agents for cleaning and sanitization.

The present invention combines the superior properties of cyclic peptides, such as the higher affinity, specificity and stability, with the ease of sequence determination by means of techniques routinely employed for linear peptides. The proposed technique is universal, as it is valid for any peptide and for any purpose that requires peptide analysis. Also it is noted that one of the novel aspects consists in the introduction of a single additional synthetic step, which is an efficient and easily controlled reaction, it is inexpensive and does not require any additional equipment.

FIG. 36a-36g shows a general schematic for the synthesis of depsipeptides on a bead.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A method for synthesizing a cyclic peptide ligand with selectivity and affinity for a biologic of interest which comprises:
(a) synthesizing a solid-phase library of reversible cyclic heterodetic peptides;
(b) selecting a reversible cyclic heterodetic peptide that shows selectivity and affinity for the biologic of interest;
(c) linearizing and sequencing the selected reversible cyclic heterodetic peptide; and
(d) solid-phase synthesizing a cyclic peptide ligand with a sequence corresponding to the selected reversible cyclic heterodetic peptide.

2. The method of claim 1, wherein the reversible cyclic heterodetic peptide is a cyclic depsipeptide.

3. The method of claim 1, wherein the reversible cyclic heterodetic peptide is a cyclic amidine-peptide.

4. The method of claim 1, wherein a plurality of cyclic peptide ligands are synthesized.

5. A method for synthesizing a cyclic depsipeptide which comprises:
(a) coupling a protected tri-functional molecule with a plurality of protecting groups onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) cleaving a protecting group from either (i) the protected amino acid or peptide, or (ii) the tri-functional molecule so as to form a deprotected amino acid or peptide, or a deprotected tri-functional molecule coupled on the solid support;
(e) coupling a protected cleavable linker with either (iii) the deprotected amino acid or peptide, or (iv) the deprotected tri-functional molecule;
(f) cleaving the protecting group from the cleavable linker and a protecting group from either (iii) the protected amino acid or peptide, or (iv) the protected tri-functional molecule and cyclizing so as to form a cyclic depsipeptide coupled on the solid support; and
(g) cleaving any remaining protecting groups from the cyclic depsipeptide coupled on the solid support.

6. The method of claim 5, wherein a solid-phase library of cyclic depsipeptides is prepared.

7. The method of claim 6, further comprising additional step (h) wherein the library of cyclic depsipeptides on the solid support is screened to identify a cyclic depsipeptide(s) that bind to a biologic of interest.

8. The method of claim 5, further comprising additional step (h) wherein the ester bond in the cyclic depsipeptide is hydrolyzed so as to yield a linear molecule on the solid support.

9. The method of claim 5, further comprising additional step (h) wherein the cyclic depsipeptide is cleaved from the solid support.

10. The method of claim 5, further comprising additional step (h) wherein both the cyclic depsipeptide is cleaved from the solid support, and the ester bond in the cyclic depsipeptide is hydrolyzed, to yield a linear molecule.

11. The method of claim 10, wherein the linear molecule is sequenced by Edman degredation or mass spectrometry.

12. The method of claim 5, wherein the cleavable linker is either an ester forming or an ester containing cleavable linker.

13. The method of claim 12, wherein the ester forming cleavable linker is either a hydroxyl protected or a hydroxyl unprotected linker, or a monoprotected dicarboxylic acid linker.

14. The method of claim 13, wherein the hydroxyl protected ester forming cleavable linker is an N,O-protected hydroxy amino acid.

15. The method of claim 13, wherein the hydroxyl unprotected ester forming cleavable linker is a carboxyl-protected α-, β-, or γ-hydroxy acid.

16. The method of claim 13, wherein the ester forming cleavable linker is a mono-ester of a dicarboxylic acid.

17. The method of claim 15, wherein the carboxyl-protected α-hydroxy acid is a lactic acid ester.

18. The method of claim 17, wherein the lactic acid ester is an alkyl lactate or an alkenyl lactate.

19. The method of claim 12, wherein the ester containing cleavable linker is either the ester of an N α-protected amino acid and a hydroxy acid, or the ester of an amino acid and a carboxyl-protected hydroxy acid, or the ester of an N α-protected amino acid and an N α-acylated hydroxy amino acid.

20. The method of claim 5, wherein the linked protected amino acid or peptide in step (c) is reacted under suitable conditions so as to add a plurality of protected amino acids to the linked amino acid or peptide on the deprotected tri-functional molecule.

21. A method of solid-phase synthesis of a cyclic homodetic peptide that binds a biologic of interest which comprises synthesizing a plurality of cyclic depsipeptides by the method of claim 5 and further comprises additional steps
(h) selecting a cyclic depsipeptide that binds to a biologic of interest;
(i) sequencing the selected cyclic depsipeptide; and
(j) synthesizing a cyclic homodetic peptide with a sequence corresponding to the selected cyclic depsipeptide.

22. A method for synthesizing a cyclic amidine-peptide, the method comprising:
(a) coupling a protected tri-functional molecule onto a solid support under suitable conditions;
(b) cleaving a protecting group from the protected tri-functional molecule to yield a deprotected tri-functional molecule coupled on the solid support;
(c) reacting the deprotected tri-functional molecule coupled on the solid support under suitable conditions so as to link at least one protected amino acid or peptide to the tri-functional molecule;
(d) deprotecting a primary amino group from the protected amino acid or peptide and a primary amino group from the tri-functional molecule so as to form a deprotected amino group on the acid or peptide and a deprotected amino group on the tri-functional molecule coupled on the solid support;
(e) reacting a bis-imidoester linker with the primary amino group on the acid or peptide and the primary amino group on the tri-functional molecule coupled on the solid support so as to form an cyclic amidine-peptide coupled on the solid support; and
(f) cleaving any remaining protecting groups from the cyclic amidine-peptide coupled on the solid support.

24-36. (canceled)
